(19) United States
(12) Patent Application Publication
(10) Pub. No.: US 2014/0179877 A1
(43) Pub. Date: Jun. 26, 2014

(54) MACROMOLECULE CONJUGATE

(71) Applicant: Biotech IgG AB, Lund (SE)

(72) Inventors: Rune NILSSON, Lund (SE); Bengt SANDBERG, Hjarup (SE); Scott WILBUR, Edmonds, WA (US)

(21) Appl. No.: 13/945,449

(22) Filed: Jul. 18, 2013

Related U.S. Application Data
(63) Continuation of application No. 12/160,521, filed on Oct. 1, 2010, now abandoned.
(60) Provisional application No. 60/758,025, filed on Jan. 11, 2006.

(51) Int. Cl. A61K 47/48; A61K 31/704; A61K 39/395; A61K 47/34

(52) U.S. Cl. CPC A61K 47/48215; A61K 47/34; A61K 31/704; A61K 39/3955
USPC 525/418; 530/391.9; 564/153

(57) ABSTRACT

The invention relates to a macromolecule comprising a polymer central core having at least two atoms to which at least two monomers are attached forming a dendrimeric structure comprising at least three polymer bonds, at least two linear polymers (b) being bond to said polymer bonds, wherein said polymers (b) at least have terminal functional groups for cytotoxic agents and at least on extended polymer (a) having a size of at least 1 carbon atoms longer than said polymers (b) and at least a terminal functional group for a targeting agent. The invention also relates to a macromolecule conjugate as well as a macromolecule biotin conjugate comprising said macromolecule, methods to produce said macromolecules as well as kits or system comprising said macromolecules and method of treating a mammal by said macromolecules.

This application is a Continuation of U.S. Ser. No. 12/160,521, filed 1 Oct. 2010, which is a National Stage application of PCT/EP2007/000209, filed 11 Jan. 2007, which claims benefit of U.S. Ser. No. 60/758,025, filed 11 Jan. 2006 and which applications are incorporated herein by reference. To the extent appropriate, a claim of priority is made to each of the above disclosed applications.

### FIELD OF INVENTION

The invention relates to a macromolecule comprising a polymer central core having at least two atoms to which at least two monomers are attached forming a dendrimeric structure comprising at least three functional groups, at least two linear polymers (b) being bond to said functional groups, wherein said polymers (b) at least have terminal functional groups for cytotoxic agents and one extended polymer (a) being at least 1 atom longer than said polymers (b) and having one terminal functional group for a targeting agent. The invention also relates to a macromolecule conjugate as well as a macromolecule biotin conjugate comprising said macromolecule, methods to produce said macromolecules as well as kits or systems comprising said macromolecules, use of macromolecules to conjugating to targeting agents and method of treating a mammal by said macromolecules. The invention further relates to means of improving the conditions for specific release of cytotoxins from a targeting agent after endocytosis.

### BACKGROUND OF INVENTION

Many biomolecules, including proteins and peptides, hold potential as reagents for use in diagnosis and therapy of human conditions and diseases. As most biomolecules do not, by themselves, have properties to make them useful as diagnostic and/or therapeutic reagents, biomolecules of interest are often chemically modified to achieve this. Modification of a targeting biomolecule with an effector agent, such as a cytotoxic agent, can provide valuable new tools for diagnosis and therapy of human and animal diseases or conditions.

Tissue or organ specific localization of a medical agent is a very important factor in its effective application. Lack of specific tissue localization is of particular importance in the treatment with medical agents where the desired effect is to kill certain types of cells such as in the treatment of cancer. In order to enhance the specificity, tumor specific monoclonal antibodies are used as a carrier (immunoconjugates) of various cytotoxic moieties, such as, but not limited to, radio nuclides, chemotherapy drugs, synthetic or natural occurring cytotoxic agents, immunosuppressive agents, immunostimulating agents and enzymes used in prodrug protocols.

Tumor-specific immunoconjugates are selectively bound to tumor cells, where an initial high concentration of the cell-toxic immunoconjugate in the blood circulation is necessary to reach a sufficient high concentration of the target tissue in a patient. While required for optimal therapy of the cancer, the high concentration of cytotoxic material in the blood and other non-tumor tissue, in most cases leads to tissue damage and/or lesion formation in sensitive and vital tissues like the bone marrow. The most effective method for preventing tissue and bone marrow damage from toxic materials in the blood is to dramatically decrease the amount of that toxic material in the blood but still retaining the therapeutic level of toxic material in the tissue being treated (e.g. tumor).

Mitra Medical AB, Lund, Sweden has developed a series of novel water soluble structures (Tag-reagent; MitraTag™) for the conjugation of biomolecules (disclosed in WO2005051424, WO0002050 and WO 01/95857). These trifunctional reagents comprise an affinity ligand via a linker, an effector agent via an optional linker and a biomolecule reactive moiety via an optional linker for the bonding of a biomolecule to the reagent. These reagents enables simultaneous and site specific conjugation of, for example a cytotoxic agent or chelating groups (for radiolabelling) and an affinity ligand (e.g. biotin). One of the advantages of using these trifunctional reagents for simultaneous conjugation of an effector ligand and an affinity ligand to a biomolecules (e.g. targeting agent) is that it render to a more homogenous population of modified biomolecules with defined ratio of affinity ligand to effector ligand, normally one to one.

These trifunctional reagents can be used in conjunction with an extracorporeal technique for clearance of the reagent from blood circulation during therapy. The affinity ligand (e.g. biotin) of the trifunctional reagents is bound to an avidin-based adsorbent on a column matrix. The technique enables processing of whole blood.

The device MitraDep®, developed and manufactured by Mitra Medical AB, Lund, Sweden, is based on this technology (EP 0 567 514 and U.S. Pat. No. 6,251,394). By using the avidin-coated filter in conjunction with biotin labelled biomolecules, the blood clearance technique can be applied equally well for e.g. chimeric or fully humanised antibodies. Clinical data reveal that during a three-hour adsorption procedure, more than 90 per cent of the circulating biotinylated antibodies can be removed by the MitraDep® system (Cancer Biotherapy and Radiopharmaceuticals vol 20 number 4, page 457-466, 2005). A number of publications provide data showing that this technique is both efficient and practical for the clearance of biotinylated and radionuclide labeled tumor specific antibodies (Norrgren K. et al, Antibody Immunoconj. Radiopharm. 4:54 (1991), Norrgren K. et al J. Nucl. Med 34:448-454 (1993); Garkavij M. et al Acta Oncologica 53:309-312 (1996); Garkavij M. et al, J. Nucl. Med. 38:895-901 (1997)).

The issue of combining an affinity reagent and effector agent on one molecule to achieve minimal modification of biomolecules is not unique to biotin as the affinity ligand or toxin or radionuclide binding/bonding moieties as the effector agent (cytotoxic agent), and is not limited to only one affinity ligand and one effector ligand per molecule.

Polyamino amide dendrimers (PAMAM) represent a new class of symmetrical highly branched spherical polymers that are highly soluble in aqueous solutions. Dendrimers have a high degree of molecular uniformity, narrow molecular weight distribution, specific size and shape characteristics, and with a surface, which may comprise primary amino groups. The manufacturing process is a series of repetitive steps starting with a central initiator core. Each subsequent growth step represents a new generation of polymer with a larger molecular diameter, twice the number of reactive surface sites, and approximately the double molecular weight of the preceding generation.

Dendritic poly (amino acid) polymer carriers with multiple functional groups at the polymer surface have been designed for the application of drug or diagnostic agent attachment. The polymer carriers are designed to permit sufficient preservation of the binding affinity of the targeting ligand while conjugating therapeutic or diagnostic agents to the polymers (WO2003055935, US 2003/0232968). Disclosed in WO2004045647 is a delivery system for the amplification of active substance delivery for drug, peptide and protein pharmaceuticals using a biotin-mediated uptake system, including the use of dendritic polymers.

In WO94/20089 taxol-based compositions with enhanced bioactivity have been disclosed. The invention includes biologically active taxol-compositions modified with e.g. poly ethylene glycol (PEG) via a carbamate linker. The attachment of the polymeric materials to the taxol increases the solubility and reduces the immunogenicity. In WO02/087497 paclitaxel, as an example of chemotherapeutic agent, is conjugated to a polymer carrier via a PEG spacer for the selective delivery of therapeutic agents to tumors or other tissues expressing biological receptors.

Poly ethylene glycol (PEG) has been conjugated to dendrimers as a delivery system for genetic material where the PEG moiety protects the dendrimer from the mononuclear phagocyte system of the organism (WO2004072153). Additional applications including PEGylated dendrimers are the use as an internal standard in mass spectroscopy for the quantification of analytes (WO2005031304).

In U.S. Pat. No. 5,830,986 a method to synthesize functionalizable poly (ethylene oxide) star molecules is disclosed. The poly (ethylene oxide) star molecules may be functionalized with biological active molecules, such as antibodies, enzymes, growth factors, diagnostic agents, organic drug molecules. One star may be functionalized with more than type of biological active molecules, such as an antibody and an organic drug molecule. The PEO-linkers may have different length. If two types of linkers with different functional groups are present they are evenly distributed on the star core and present in a similar amount. Further the used PEO-linkers present are a narrow molecular distribution of PEO-linkers.

Furthermore, use of distribution of PEO-linkers is disadvantageous, as the properties of the formed structures will be a mean of the properties of individual structures. This is troublesome if the structures are intended to be used as pharmaceuticals. It may be difficult to get approval to use a mixture of structures in a pharmaceutical composition. Presence of a plurality of antibodies is disadvantageous as antibodies are large, slow diffusional structures. A structure with several antibodies will therefore need a long time to reach its target. Further is the disadvantageous to have a similar ratio of e.g. antibodies and organic drug molecules. The load of organic drug molecules per antibody is not increased compared to a situation where the organic drug is directly coupled to the antibody. Use of a hydrolysable group, i.e. different types of carboxylic acids and polycarboxylic acids, inserted in between the functional group and an organic drug molecule is also disclosed. The usefulness of carboxylic acids as a hydrolysable group is limited as they are stable at physiological conditions.

U.S. Pat. No. 6,737,236 discloses a novel method for conjugating macromolecules (e.g. protein, drugs and prodrugs) to other molecular entities using cycloaddition reactions, such as the Diels-Alder reactions. In this invention coumarin is used as a diagnostic detector molecule.

There are several cytotoxic agent delivery systems based on polymers described, but design improvements are needed. There is clearly a need to optimize therapy involving cell-killing agent, where the concept are to largely extent genetic, in so far that as many as possible of the parameter are independent on the type and localization of the disease and as much as possible independent on the pharmacokinetic parameters and rate of metabolisms of the individual patient. An improved cytotoxic agent delivery system should preferably exhibit characteristics of being effective and specific as cytotoxic agent carriers, show biocompatibility and biodegradability, being biologically stable in the blood circulation and showing reduced immunogenicity and allow the release of the cytotoxic agent in its active form after internalization. Further, there is a need for cytotoxic agent delivery systems with an increased number of cytotoxic agents per tissue specific targeting agent. As the tissue specific targeting agent will slow down the penetration of the conjugate into the tissue, it is highly disadvantageous to have a similar number of cytotoxic agents and tissue specific targeting agents in a given cytotoxic agent delivery systems.

### SUMMARY OF THE INVENTION

The invention relates to an improved macromolecule in which said macromolecule have the unique properties of being able of selectively delivering a high dose and a high number of one or more cytotoxic agents within a mammal, such as delivering a high dose of a cytotoxic agent to a cancer cell.

The invention relates in one aspect to a macromolecule comprising a polymer central core having at least two atoms to which at least two monomers are attached forming a dendrimeric structure comprising at least three functional groups, at least two linear polymers (b) being bond to said functional groups, wherein said polymers (b) at least have terminal functional groups for cytotoxic agents and one extended polymer (a) being at least 1 atom longer than said polymers (b) and having one terminal functional group for direct or indirect coupling to the targeting agent.

In another aspect, the invention relates to a macromolecule conjugate comprising a macromolecule and a targeting agent bond to said polymer (a) via said terminal functional group suitable for coupling to said targeting agent.

In a further aspect, the invention relates to a macromolecule biotin conjugate comprising a macromolecule and at least one trifunctional cross-linking moiety bond to said polymer (a), said trifunctional cross-linking moiety being coupled to a biotin molecule via linker I wherein linker I contains hydrogen bonding atoms, preferably ethers or thioethers, or ionisable groups, preferably carboxylate, sulphonates and ammonium to aid in water solubilisation of the biotin moiety, and stability against enzymatic cleavage has been provided by introducing substituents to the biotinamide amine or to a carbon atom adjacent to that amine and at least one targeting agent reactive group via linker III, wherein linker III is selected from a group comprising ethers, thioethers, or ionisable groups comprising carboxylates, sulfonates, amino, and ammonium groups.

In a further aspect the invention relates to a kit comprising a macromolecule conjugate or a macromolecule biotin conjugate and an extracorporeal device comprising at least biotin or biotin derivative binding agents.

In a further aspect the invention relates to a method for synthesising a macromolecule comprising a polymer central core having at least two arms to which at least two monomers are attached forming a dendrimeric structure comprising at least three functional groups, first provide less than one extended polymer (a) per dendrimeric structure, said extended polymer (a) having at least one terminal functional group useful for the linking, directly or via a linker, of said linear polymer (a) to the targeting agent, then provide at least two linear polymers (b) per said dendrimeric structure, said linear polymer (b) is at least one atom shorter than said extended polymer (a) and has at least have one terminal functional groups for linking cytotoxic agents, and provide means of first coupling said extended polymer (a) and then said linear polymers (b) to said dendrimeric structure to obtain said macromolecule.

In another aspect the invention relates to a method of synthesising a macromolecule conjugate by using various means of linking the macromolecule as defined above to a targeting agent as well as synthesising a macromolecule biotin conjugate as exemplified in FIG. 24 by using various means of linking a biotin moiety and optionally a detection marker to a trifunctional structure which is linked to the targeting agent, optionally linked to through a linker.

In another aspect the invention relates to the use of said macromolecule, said macromolecule conjugate, said macromolecule biotin conjugate or said kit or system for the treatment or diagnosis of mammal, such as an human being, in need thereof.

In another aspect the invention relates to the use of said macromolecule carrying suitable cytotoxic agents for the conjugation to a targeting agent.

In a another aspect the invention relates to a method of treating a mammal such as a human being suffering from a disease by administering a therapeutically effective amount of a macromolecule conjugate, a macromolecule biotin conjugate by using the kit.

Further advantages and objects with the present invention will be described in more detail, inter alia with reference to the accompanying drawings.

### BRIEF DESCRIPTION OF THE DRAWINGS

FIGS. 1 to 19 shows examples how to produce a macromolecule with or without cytotoxic agent.

FIGS. 20 to 23 shows properties of macromolecules or molecules similar to macromolecules of the invention.

FIG. 24 shows one embodiment. The numbers in FIG. 24 indicate one example of the different components of the macromolecule. However, this is solely one embodiment and the invention should not be limited thereto.

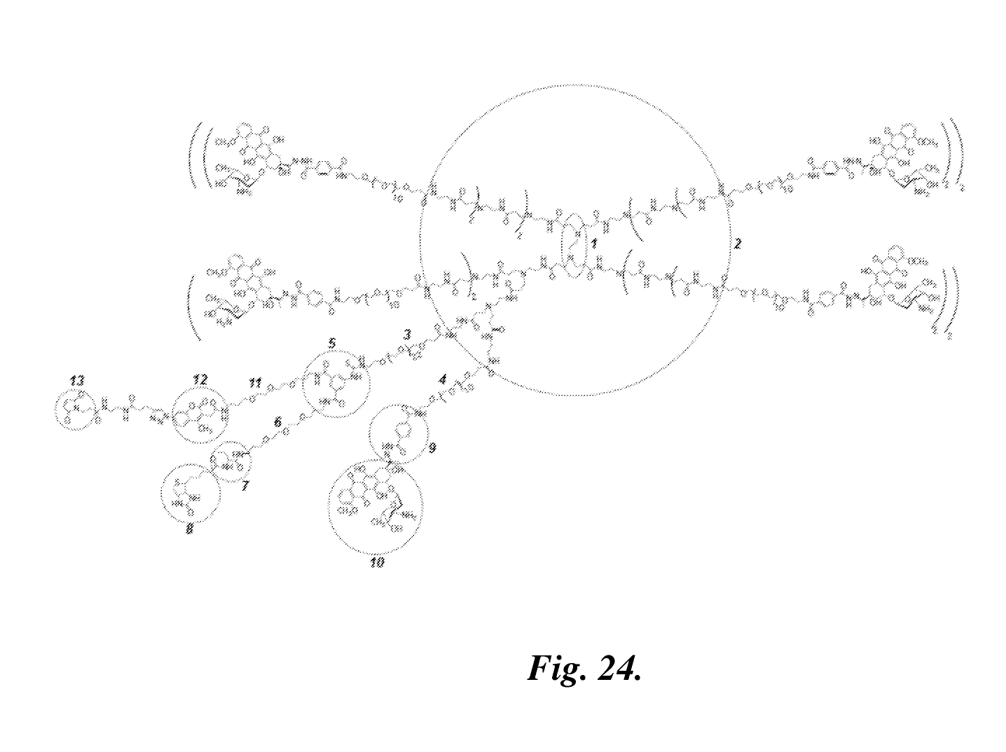

Number 1, in FIG. 24, illustrates the central core, 2 illustrates the dendrimer, 3 illustrates the polymer a, 4 illustrates the polymer b, 5 illustrates the trifunctional cross-linking unit, 6 illustrates the linker I, 7 illustrates the enzymatic protection group, in this embodiment valine, 8 illustrates the affinity ligand, in this case biotin, 9 illustrates the linker II and a degradable moiety, in this case an acylhydrazone, 10 illustrates the cytotoxic agent, in this case Daunorubicin (daunamycin), 11 illustrates linker III, 12 illustrates the detection marker, in this case a coumarate group, 13 illustrates the targeting agent reactive group.

The synthetic pathway described above is one example of one pathway to achieve one example of macromolecule as defined in this document and should not in anyway limit the scope of the invention anyone skilled in the art could easily identify alternative standard chemistry procedures for the linking the components together and/or change the order in which the various components are put together in order to end up with the identical structure of the final compound; the macromolecule.

### DETAILED DESCRIPTION OF THE INVENTION

### Definitions

In the context of the present application and invention, the following definitions apply:

The term “macromolecule” is intended to mean a polymer central core having at least a first layer of monomers attached to said central core defining a dendrimeric structure and a number of polymer arms to which cytotoxic agents may be linked via terminal functional groups, optionally via a linker II, as well as having one terminal reactive functional group for the linking to a targeting agent directly or via a trifunctional cross-linking group (see FIG. 24, numbers 1, 2, 3, 4, 9 and 10). In some part of this document a macromolecule may also be defined as the above structure lacking the reactive functional group or a residual part of the macromolecule after the macromolecule has been linked to a targeting agent either directly or via a cross-linking group defined above.

The term “macromolecule conjugate” is intended to mean a macromolecule as defined above wherein one or more cytotoxic agents are bond to said terminal functional groups for cytotoxic agent and one targeting agent is bond to said terminal binding site for targeting agents (for one specific example see FIG. 24, see FIG. 24, numbers 1, 2, 3, 4, 9 and 10).

The term “macromolecule biotin conjugate” is intended to mean a macromolecule as defined above wherein one or more cytotoxic agents are bond to said terminal functional groups for cytotoxic agents and a trifunctional cross linking moiety bound to said terminal binding site for targeting agents, wherein said trifunctional cross linking moiety is bound to a biotin moiety via linker I and one targeting agent optionally via linker III (see FIG. 24, numbers 1, 2, 3, 4, 9, 10, 5, 6, 7, 8, 11, 12 and 13).

The term “cytotoxic agent” is intended to mean an agent, which is capable of reducing the number of cells and/or eliminating the cells that the targeting agent is directed against. Said cytotoxic agent may be any kind of compound as long as the compound reduces the number of target cells and or eliminate the target cells to be affected. The cytotoxic agent may assert its effect by acting on the vital cell function or through irradiation. Examples of cytotoxic agents can be found in the following description.

The term “targeting agent” is intended to mean any agent that is capable of specifically targeting to specific cells within a mammal such as a human being. Examples are antibodies, such as monoclonal antibodies, vitamins, such as vitamin D, hormones, neurotransmitters, proteins and peptides and parts thereof, synthetic and semisynthetic variants thereof. The antibody may also be an antibody fragment such as F(ab′)₂, F(ab′), 2Fab′, F/ab), genetically engineered hybrids such as a humanised or a chimeric antibody or chemically synthesised peptides.

The term “target” is intended to mean any structural element that the targeting agent is directed against and to which the targeting agent specifically interacts and thereby provides selectivity or preference between neoplastic tissue or cells and healthy tissue or cells. Examples are molecules, which are produced in high amounts, such as cell surface structures over-expressed on different cancer cells and neovascular structural elements regulating angiogeneses. Preferences is given to targets where the macromolecure linked to the targeting agent can be internalized and thereby release the cytotoxic agents inside the target cells. It should be stressed, however, that part of the reduction of the undesirable cells to be target may be achieved through extracellular release of cytotoxic agents in the vicinity of such cells or by intracellularly released cytotoxic agents leaking out from the target cells, so called by-standard effect. In the case where the cytotoxic agent is a radio nuclide the is no requirement for internalisation.

The term “cancer” is intended to mean a neoplastic disease regardless of its histological origin. Examples of cancers includes, but is not limited to, haematological cancer such as, but not limited to various types of leucemia, lymphoma and multiple myeloma as well as solid tumours, such as, but not limited to, breast cancer, ovarian cancer, colon cancer, lung cancer, head cancer, neck cancer, CNS tumour, prostate cancer, bone cancer and liver cancer.

The term “detection marker” is intended to mean a compound comprising a structural element that absorbs or emits UV and/or visible light or emits radiation, such as but not limited to gamma-radiation. These structural elements may be fluorescent, chemiluminescent, or radioactive. Examples thereof are chromophores, bioluminescent compounds or diagnostic detector molecules, such as maleimide derivatised fluorescein, coumarin and/or radionuclides such as, but not limited to, radiohalides or a metal chelators carrying radionuclides.

A Macromolecule

In one embodiment, the invention relates to a macromolecule comprising a polymer central core, which constitutes of at least two atoms, to which at least two monomers are attached. Said monomers forming a first layer of monomers, defined as generation 0, and said central core and said first layer of monomers forming a dendrimeric structure. Said first layer of monomers comprises at least three functional groups, to which at least two linear polymers (b) and a third single extended linear polymer (a) are bound, wherein said at least two linear polymers (b) carry cytotoxic agents or at least have terminal functional groups suitable for coupling to a cytotoxic agent, optionally via linker II, and said third single extended linear polymer (a), being least 1 atom longer than said linear polymer (b), and are linked to one targeting agent or have a terminal reactive functional group suitable for direct or indirect coupling to a targeting agent. The macromolecule when bound to a targeting agent, i.e. a macromolecule conjugate or a macromolecule biotin conjugate, have unique properties such as being able to bind selectively to a target, such as to specific target cell, for example a tumour cell and deliver a high dose, to said cell of for example a cytotoxic agent and thereby being able of reducing the number of target cells and/or destroying said cells. By increasing the dose of the cytotoxic agent on a macromolecule conjugate or a macromolecule biotin conjugate, it is possible to deliver a high dose of toxic payload per targeting agent. By the use of a dendrimeric structure, which may be expanded to become a large dendrimeric complex, it is possible to increase the amount of a cytotoxic agent that should be delivered to a target. An enhanced dose per macromolecule conjugate or macromolecule biotin conjugate is achieved by providing an increased number of arms to which cytotoxic agents is linked and only having one extended arm which are linked to one targeting agent or have a terminal reactive functional group suitable for coupling a to one targeting agent. For example a less potent cytotoxic agent may be administrated in even larger amounts if a larger dendrimeric complex is used. Because of the limited rate of tissue penetration for most targeting agents (e.g. antibodies) it is of vital importance to be able to deliver a sufficient number of cytotoxic agents even in poorly vascularized part of the tumour.

The polymer layer forming a dendrimeric structure may be extended by a second layer forming generation 1 being bond to the first monomer layer etc.

The dendrimeric structure may have from 4 to 384 branches by forming expanding monomer layers, such as having 4, 8, 16, 32, 64, 128 or 256 branches or 6, 12, 24, 48, 96, 192 or 384, wherein each 4 or 6 branches defines a first layer, 8 or 12 branches a second layer etc. The dendrimeric structure may have molecular weight from about 600 to about 60 000 Da, such as about 3 000, 7 000, 14 000, 29 000 or 59 000 and may be in the form of a star like symmetrical structure. Examples of dendrimeric structures are diaminobutanepoly(propylene imino) DAB or PAMAM. The dendrimeric structure may comprise linear polymer structures as well as branched polymer structures. The linear polymers (a) and (b), which are attached to said dendrimeric structure, may be selected from the group consisting of polyamino acid, such as polyglycine, polytyrosine and polyphenylalanine, dextran, polysaccharides, polypropylene oxide (PPO), poly D-amino acids, a copolymer of polyethylene glycol (PEG) with PPO, PEG, polyglycolic acid, polyvinyl pyrolidone, polylactic acid and polyvinylalcohol or a mixture thereof. The linear polymer structures are therefore, in one embodiment of the present invention, hydrophilic. A hydrophilic polymer structure provides the macromolecule with increased water solubility. Such a hydrophilic polymer structure may also counteract the decreased water solubility, which hydrophobic cytotoxic agents may contribute to. Further, a hydrophilic polymer may prevent hydrophobic cytotoxic agents on the same macromolecule conjugate from aggregating, or at least reduce such aggregation. Said extended linear polymer (a) have an extended length, compared to the other linear polymers, to separate the targeting agent from the branched central core and thereby enable the possibility for the targeting agent to bind in a selectively and efficient way to said target. The extended linear polymer (a) may have a size wherein the length ratio of polymers (b) and the extended polymer (a) may be from about 1:1.2 to about 1:4, such as from about 1:1.5 to about 1:3 or from about 1:1.5 to about 1:2.5. The length of said extended linear polymer (a) may be about 11 to about 200 atoms, i.e., the backbone atoms in the linear polymer, such as 25, 30, 40, 50, 60, 70, 80, 90, 100 atoms. The linear polymers (b), which always are at least 2, may have equal length or being different and have a length about 10 to about 100 atoms, such as 20, 30, 40, 50, 60, 70, 80 or 90 atoms. However, the extended linear polymer (a) should be at least 10% longer than the other linear polymers, such as 20, 30, 40, 50, 60, 70, 80, 90, 100 or even 200% longer. The group linking the linear polymers to said dendrimeric structure may be selected from the group consisting of amides, carboxylic acid esters, thioesters, disulfides, thiourethanes, carbamates, carbonates, thioureas or ureas.

In one embodiment the polymers comprise discrete PEG polymers. The size of the discrete PEG polymer in polymer (a) is then at least 12 repeating —CH2-CH2-O— units. The size of discrete PEG polymer in the polymer (b) is then at least 8 repeating —CH2-CH2-O— units. The linear polymers may be linked to said dendrimeric structure via an amide bond. An amide bond can be formed in several ways, as is well known in the art, and is, once formed, a stable bond not easily hydrolyzed under neither acidic nor basic conditions. In such an embodiment the extended polymer (a) is at least 2 repeating —CH2-CH2-O— units longer than the polymer (b). Use of discrete PEG polymers has the advantage of giving a structure a defined size and shape. It will also be easier to get approval for use as a medicament if the structure consists of discrete structural elements rather than elements from a distribution, e.g. a narrow distribution of PEO-polymers. If a structure comprises a distribution of parts, such as PEO-polymers, the properties may vary from time to time depending on the exact distribution, this means that the properties of the structures are very hard to predict.

In another embodiment the extended polymer (a) comprises 24 repeating —CH2-CH2-O— units and polymer (b) comprises 12 repeating —CH2-CH2-O— units.

The linear polymers (b) have at least one terminal functional group suitable for coupling to at least one cytotoxic agent, optionally via a linker II. Said terminal functional group of said linear polymer (b) or linker II may be a group selected from the group consisting of an amine group, a hydroxyl group, a carboxyl group, such as an carboxylic acid, amide, carboxylic halide, carboxylic acid ester or carboxylic acid anhydride, said carboxyl group may be activated, as is well known in the art, to facilitate coupling, a sulfhydryl group, a alkyne, an azide, a vinylsulfone group, a maleimide group, an isothiocyanate group, isocyanate group or a hydrazine group, such as an allylhydrazine, alkylacylhydrazine, arylhydrazine or arylacylhydrazine.

In one embodiment the linear polymers (b) are coupled to a linker II, wherein linker II carries a functional group which enables coupling of linker II to said cytotoxic agent.

Said terminal functional groups may be the same or different and may bind to one and the same cytotoxic agent or to different cytotoxic agents.

Said linker II may include a degradable, biodegradable or releasable moiety and may be cleavable upon change of pH, change in the redox potential, reduction of a cystin residue or other forms of disulphide bridges, electrophilic or nucleophilic attack or by an enzymatic process. Examples of acid labile groups are carbamates, thiocarbamates, carbonate groups, thiocarbonate groups, ureas, thioureashydrazones or Cis-aconityls and derivatives thereof being acid labile. The advantages of using carbamates, thiocarbamates, carbonate groups, thiocarbonate groups, ureas, thioureashydrazones or Cis-aconityls and derivatives thereof being acid labile as a linker is its stability in aqueous based systems at neutral pH, while it is hydrolyzed under acidic condition such as the condition within the endosomes and/or lysosomes, after being internalized through endocytosis.

In another embodiment the moiety, which links the cytotoxic agent to the macromolecule, in itself constitutes a degradable, biodegradable or releasable moiety which may be cleavable upon change of pH, change in the redox potential, reduction of a cystin residue or other forms of disulphide bridges, electrophilic or nucleophilic attack or by an enzymatic process.

In another embodiment such a moiety, which links the cytotoxic agent to the macromolecule, is an acid labile group such as a carbamate, a carbonate, or hydrazone. Some advantages of using an acid labile moiety to link the cytotoxic agent to the macromolecule have been discussed above. It is further advantageous to have a degradable moiety to link the cytotoxic agent to the macromolecule, as this means that the cytotoxic agent may be released in its native form, active form. Further, by altering the substitutents on the carbamate or hydrazone it is possible to tune the acid lability and even customize it depending on the specific toxic agent used. Such modifications are well known to the one skilled in the art.

In another embodiment linker II is bound to polymer (b) via an acid labile group, such as have been described above, and the cytostatic agent via second group. This second group may be cleaved by the remaining part of the acid labile group, eg. by a nucleophilic attack.

By introducing a degradable, biodegradable or releasable linker II or moiety, which links the cytotoxic agent to the macromolecule, the cytotoxic agent may be released at the target site or in the target cell depending on the linker or moiety used. The target, to which the targeting agent is directed, may facilitate an internalisation process of the bound macromolecule conjugate/macromolecule biotin conjugate. By enabling the possibility to release the cytotoxic agent inside the target cell the potency of said cytotoxic agent may be increased. The linker and/or moiety shall be stable in blood circulation in vivo for an extended period of time as well as during storage prior to administration. In one embodiment the linker and/or moiety shall be readily cleavable inside target cells after the macromolecule conjugate/macromolecule biotin conjugate is internalized via endocytosis and where the cleavage to release the cytotoxic agent may occur primarily in primary or secondary endosomes ore in matured endosomes such as the lysosomes.

If the linker and/or moiety are acid labile, it will be stable at neutral pH, as in the blood, but could be hydrolyzed at lower pH such as in tumor tissue or inside primary or secondary endosomes or in matured endosomes such as the lysosomes, i.e. nearby or within the target cells.

Said cytotoxic agent may be a natural or synthetic agent acting at different mechanism such as inhibiting DNA or RNA synthesis, inhibiting protein synthesis or interaction with tubulin, topoisomerase inhibitors, ionophores and interaction with heat shock proteins and may come from various natural sources like bacteria, plants or animals. Examples of cytotoxic agents are: taxanes, such as Taxotere® and Taxol®, Vinblastine, Vincristine, desacetyl vinblastine, desacetyl vinblastine hydrazine, daunorubicin, geldanamycin, ricin, abrin, diphtheria toxin, modecin, tetanus toxin, mycotoxins, mellitin, α-amanitin, pokeweed antiviral protein, ribosome inhibiting proteins, auristatin E, auristatin EB (AEB), auristatin EFP (AEFP), monomethyl auristatin E (MMAE), 5-benzoylvaleric acid-AE ester (AEVB), tubulysins, disorazole, epothilones, SN-38, topotecan, rhizoxin, duocarmycin, actinomycin, ansamitocin-P3, duocarmycin, duocarmycin B2, maytansine, maytensinoids (DM1, DM2, DM3, DM4), calicheamicin, echinomycin, colchicine, estramustine, cemadotin, eleutherobin, 1-hydroxyauramycin A, aclacinomycin Abafilomycin C1, dinaktin, doxorubicin and doxorubicin derivatives such as morpholino-doxorubicin and cyanomorpholino-doxorubicin, dolastatin such as dolestatin-10, combretastatin, leptomycin B, pluramycins, staurosporine, nogalamycin, rhodomycins, mithramycin, rabelomycin, rapamycin, alnumycin, chartreusin, geliomycin, gilvocarcin, piericidin, chlorambucil, cyclophosphamide, melphalan, and cyclopropane and antimetablites such as methotrexate, dichlorormethatrexate, methopterin, cytosine arabinoside, leurosine, leurosideine, mitomycin C, mitomycin A, carminomycin, aminopterin, tallysomycin, podophyllotoxin, camptothecin, cisplatin, carboplatin, and metallopeptides containing platinum, copper, vanadium, iron, cobolt, gold, cadmium, gallium, iron zinc and nickel or radionuclides, such as α,β or gamma-radiation. However, other known drugs may be modified in order to provide a functional group for conjugation to the linker described herein. Such chemical modification is known in the art.

The linear extended polymer (a) has a terminal functional group for direct or indirect coupling a targeting agent, wherein said terminal functional is selected from the group consisting of an amine group, a hydroxyl group, a carboxyl group, such as an carboxylic acid, amide, carboxylic halide, carboxylic acid ester or carboxylic acid anhydride, said carboxyl group may be activated, as is well known in the art, to facilitate coupling, a sulfhydryl group, a vinylsulfone group, alkyne group, azide group, a maleimide group, an isothiocyanate group, an isocyanate group, an imidate group, alpha-halo-amide, Michael acceptor, an hydrazide group, an oxyamine group or a combination thereof.

In another embodiment the terminal functional group for coupling a targeting agent comprise a group useful in “click-chemistry”, such as an azide or alkyne. Use of “click-chemistry” to couple the targeting agent to the macromolecule is advantageous as the conditions and reagents used in a such a coupling is very mild and may not affect other parts of the macromolecule, such as the cytotoxic agent or the group linking it to the macromolecule.

Additionally, at least one of the polymers in (b) and/or (a) may comprise a detection marker. By providing a detection marker it is possible to determine how many macromolecules are conjugated to each targeting agent and thereby the ratio of targeting agent on one hand and the number of biotin residues, cytostatic agents on the other hand. A detection marker also makes it possible to determine the amount of macromolecule conjugate or macromolecule biotin conjugate present at the target. Detection markers may be compounds comprising structural elements selected from the group of structural elements that absorb or emit UV and/or visible light or emit radiation, such as but no limited to gamma-radiation. The elements may be fluorescent, chemiluminescent, or radioactive. Examples thereof are chromophores, bioluminescent compounds or diagnostic detector molecules, such as maleimide derivatised fluorescein, coumarin or a metal chelator. Further presence of detection marker such as a group that emits emit radiation, such as but no limited to gamma-radiation, may also make it possible to trace the macromolecule, if injected into a animal, such as a mammal, such as an human being. A detection marker may also make it easy to quantify the macromolecule in fluids, such plasma or blood.

A Macromolecule Conjugate

In another embodiment, the invention relates to a macromolecule conjugate comprises said macromolecule mentioned and defined above and a targeting agent as defined above. The targeting agent being coupled via said terminal functional group. If said targeting agent is an antibody it may be selected from the group consisting of polyclonal, monoclonal antibodies, semisynthetic or synthetic variants thereof or parts of monoclonal antibodies Examples of antibodies, which can be conjugated to said macromolecule, are any antibody, part or combination thereof as long as the antibody, part or combination has the ability to bind to a target.

Essentially all of the macromolecule conjugates will contain only a single targeting agent. The presence of more than one targeting agent does not increase the affinity towards the target substantially significantly, but it does slow down the tissue penetration significantly, due to the size of the targeting agent. A slow tissue penetration means that the exposure of parts outside the target, for the cytotoxic agent, may get unnecessary high, as the time needed for the macromolecule conjugate to reach the target is extended.

A macromolecule conjugate may contain more than one macromolecule per targeting agent. Further, it is not necessary for all of the targeting agents to have the same number of macromolecules bound to them. The predominant number of the macromolecules per targeting agent in the macromolecule conjugate may range from 1/1 to 6/1 such as 1/1, 2/1, 3/1, 4/1, 5/1 or 6/1. The average number of macromolecule residues per number of targeting agent in a preparation of macromolecule conjugates may range from less than 1 to 6 such as 0.5 to 6.0, such as 1.0 to 4.0, 1.0 to 3.0 or 1.0 to 2.0, such as 2.0 to 6.0 or 2.0 to 4.0, such as 3.0 to 6.0 or 3.0 to 4.5, such as 4.0 to 6.0 or 4.0 to 5.0, such as 5.0 to 6.0. If the preparation comprise targeting agents not bound to any macromolecule it may be necessary to remove them before use of the macromolecule conjugate.

A Macromolecule Biotin Conjugate

In a another embodiment the invention relates to a macromolecule biotin conjugate, which comprises said macromolecule conjugate mentioned and defined above and a trifunctional cross-linking moiety selected from the group comprising of triaminobenzene, tricarboxybenzene, dicarboxyaniline and diaminobenzoic acid coupled to said extended linear polymer (a), said trifunctional cross-linking moiety being coupled to a biotin molecule via linker I and to a targeting agent reactive group, optionally via linker III. The biotin molecule is selected from the group consisting of biotin derivatives selected from the group comprising norbiotin, homobiotin, diaminobiotin, biotin sulfoxide, biotin sulfone or other biotin molecules having the ability to bind to and having essentially the same binding function to avidin or streptavidin as biotin such as having an affinity constant of ≧10⁶, such as 10⁷, 10⁸, 10⁹, 10¹⁰, 10¹¹, 10¹², 10¹³, 10¹⁴, 10¹⁵ and coupled to said trifunctional cross-linking moiety via a linker I, wherein the linker I contains hydrogen bonding atoms, preferably ethers or thioethers, or ionisable groups, preferably carboxylate, sulphonates, amino and ammonium groups to aid in water solubilisation of the biotin moiety, and stability against enzymatic cleavage has been provided by introducing substituents to the biotinamide amine or to an carbon atom adjacent to that amine, such as by the introduction of an alkyl group such as an alpha or beta group, wherein said alkyl group comprises 1 to 5 carbon atoms, being linear or branched and may include carboxyl, carboxy amide or hydroxyl groups (such as alpha or beta aspartyl, aminobutyric acid, serine, threonine, valine etc.)

Said targeting agent reactive group can be coupled to a targeting agent as defined above. Said targeting agent reactive group may be coupled to said trifunctional cross-linking moiety via a linker III, wherein linker III contains ethers, thioethers, or ionisable groups comprising carboxylates, sulfonates, amino and ammonium groups.

As have been described above, it is important that the essentially all of macromolecule are bound only to a single targeting agent. This is achieved by only having one extended polymer (a) on each macromolecule and only one targeting agent reactive group on the extended polymer (a).

A macromolecule biotin conjugate may contain more than one macromolecule per targeting agent. Further, it is not necessary for all of the targeting agents to have the same number of macromolecules bound to them. The predominant number of the macromolecules per targeting agent in the macromolecule conjugate may range from 1/1 to 6/1 such as 1/1, 2/1, 3/1, 4/1, 5/1 or 6/1. The average number of macromolecule residues per number of targeting agent in a preparation of macromolecule conjugates may range from less than 1 to 6 such as 0.5 to 6.0, such as 1.0 to 4.0, 1.0 to 3.0 or 1.0 to 2.0, such as 2.0 to 6.0 or 2.0 to 4.0, such as 3.0 to 6.0 or 3.0 to 4.5, such as 4.0 to 6.0 or 4.0 to 5.0, such as 5.0 to 6.0.

In the macromolecule biotin conjugate the length of the extended polymer (a) is important, for the binding of said targeting agent to said target and enable the possibility of removing macromolecule biotin conjugates that have not been bond to any target from a body fluid such as blood. The macromolecule biotin conjugate may be removed from the body fluid by the use of an extracorporeal device comprising avidin or streptavidin as the binding agent for the biotin molecule. However, if the extended polymer (a) is to short, steric hindrance occur and the macromolecule biotin conjugate cannot be removed from the body fluid.

The linkers I and III in said macromolecule biotin conjugate may have the same or different length. Linker I may have a length of at least 5 atoms, such as from 5 to about 50 atoms and linker III may have a length of from 1-25 atoms, such as from about 5 to about atoms. Additionally linker III may comprise a detection marker, such detection markers and the advantages of incorporating them have been described above.

Antibodies that can be conjugated to said macromolecule is any antibody or part thereof as long as the antibody or part has the ability to bind to a target.

A Pharmaceutical Composition Comprising a Macromolecule Conjugate and/or a Macromolecule Biotin Conjugate

A pharmaceutical composition, which comprises a macromolecule conjugate or macromolecule biotin conjugate, may further comprise pharmaceutically acceptable carriers, diluents, stabilisers or excipients.

“Pharmaceutically acceptable” means a carrier, stabiliser, diluent or excipient that at the dosage and concentrations employed does not cause any unwanted effects in the patients to whom it is administered. Such pharmaceutically acceptable carriers or excipients are well-known in the art (see Remington's Pharmaceutical Sciences, 18th edition, A.R Gennaro, Ed., Mack Publishing Company (1990) and handbook of Pharmaceutical Excipients, 3rd edition, A. Kibbe, Ed., Pharmaceutical Press (2000). The compositions according to the invention may be lyophilised.

The pharmaceutical compositions may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

The pharmaceutical composition according to the invention may be administered intravenously, intraperitonealy or intratumourly. Suitable liquid pharmaceutical preparation forms are, for example injectable solution in ampule form and also preparations with protracted release of active compounds, in the preparation of excipients, diluents, adjuvants or carriers are customarily used as described above.

The pharmaceutical composition will be administered to a patient in a pharmaceutically effective dose. By “pharmaceutically effective dose” is meant a dose that is sufficient to produce the desired effects in relation to the condition for which it is administered. The exact dose is dependent on the activity of the compound, manner of administration, nature and severity of the disorder, the patients general conditions, age and body weight of the patient and different doses may be needed.

The pharmaceutical composition of the invention may be administered alone or in combination with other therapeutic agents. These agents may be incorporated as part of the same pharmaceutical composition or may be administered separately.

The pharmaceutical compositions may be administered once as a single dose or repeatedly over a certain period of time. The pharmaceutical composition may be administered 3 times a month or 4 times a month.

A Kit or a System

The invention also relates to a kit, system or a method of using said kit or system, comprising the macromolecule, macromolecule conjugate or macromolecule biotin conjugate mentioned and defined above and an extracorporeal device comprising at least biotin or biotin derivative binding agents, such as avidin or streptavidin or derivatives thereof. One example of such an extracorporeal device is Mitra-Dep®. By providing such a kit it is possible to use the kit for the removal of conjugates which have not bound the target in a mammal such as a human, dog, cat, horse, cow, camel or any other animal by introducing said macromolecule, macromolecule conjugate, macromolecule biotin conjugate into the mammal, allowing said macromolecule, macromolecule conjugate, macromolecule biotin conjugate to circulate within said mammal and binding to said target, and finally allowing the body fluid passing the extracorporeal device and allowing binding of free conjugates to said extracorporeal device and thereby enable the removal of the surplus of macromolecules, macromolecule conjugate, macromolecule biotin conjugate from the mammal. By removing the surplus of macromolecules, macromolecule conjugate, macromolecule biotin conjugate from cells and organs within the mammal which the macromolecule are not directed against the exposure to these healthy cells and organs will be diminished. Said biotin or biotin derivative binding agents are avidin or streptavidin or derivatives thereof and a device coated with these or alike binding agents are utilized. Such a device could be a filter (e.g. holofiber filter) or filled with an adsorbent.

As the macromolecule conjugate or macromolecule biotin conjugate mentioned and defined above comprises only one targeting agent, the time needed for the conjugate to reach the target is shorter than if more than one targeting agent would have been present. This means that time of exposure to healthy cells and organs, within the mammal, which the macromolecule are not directed against, is reduced and thereby are the side effects reduced.

For such affinity adsorbents, the matrix may be of various shapes and chemical compositions. It may for example constitute a column house filled with particulate polymers, the latter of natural origin or artificially made. The particles may be macroporous or their surface may be grafted, the latter in order to enlarge the surface area. The particles may be spherical or granulated and be based on polysaccharides, ceramic material, glass, silica, plastic, such as, but not limited to, polyvinyl alcohol (PVA)-cryogel beads and dimethyl acrylamide (DMAAm) monolithic or any combination of these or similar material. A combination of these could, for example, be solid particles coated with a suitable polymer of natural origin or artificially made. Artificial membranes may also be used. These may be flat sheet membranes made of cellulose, polyamide, polysulfone, polypropylene or other types of material which are sufficiently inert, biocompatible, non-toxic and to which the receptor could be immobilized either directly or after chemical modification of the membrane surface. Capillary membranes like the hollow fibers made from cellulose, polypropylene or other materials suitable for this type of membranes may also be used. A preferred embodiment is a particulate material based on agarose and suitable for extracorporeal extraction.

The kit or system may also comprise one or more tubings. The tubing set is/are adapted to the extracorporeal device as well as the apparatus to be used to remove the whole blood from the mammal. Examples of such tube sets are the tube set from Fresenius Medical Care AG, D-61346 Bad Hamburg, Del., inlet tubing line “Art” art no: 9798814 and return tubing line, art no: 979521-1.

To facilitate the extracorporeal depletion an apparatus for extracorporeal circulation of whole blood or plasma will be connected to the patient through tubing lines and blood access device(s). Such an apparatus should provide conduits for transporting the blood to an adsorption device and conduits for returning the processed blood or plasma to the patient. In the case plasma is processed through the adsorption device, a plasma separation device is needed as well as means of mixing the concentrated blood with processed plasma. The later is normally achieved by leading the two components into an air-trap where the mixing occurs. Any person skilled in the art of extracorporeal technology would be familiar with the wide range of equipment and disposables available for that purpose.

In the case where whole blood is processed, an ordinary dialysis machine can constitute the base for such an apparatus. Dialysis machines are normally equipped with all the necessary safeguards and monitoring devices to meet patient safety requirements and allow easy handling of the system. Hence, in a one embodiment whole blood is processed and a standard dialysis machine is utilised with only minor modifications of the hardware. However, such a machine requires a new program fitted to the new intended purpose.

Blood access could be achieved through peripheral vein catheters, or if higher blood flow is needed, through central vein catheters such as, but not limited to, subclavian or femoral catheters.

A Method how to Produce Said Macromolecule

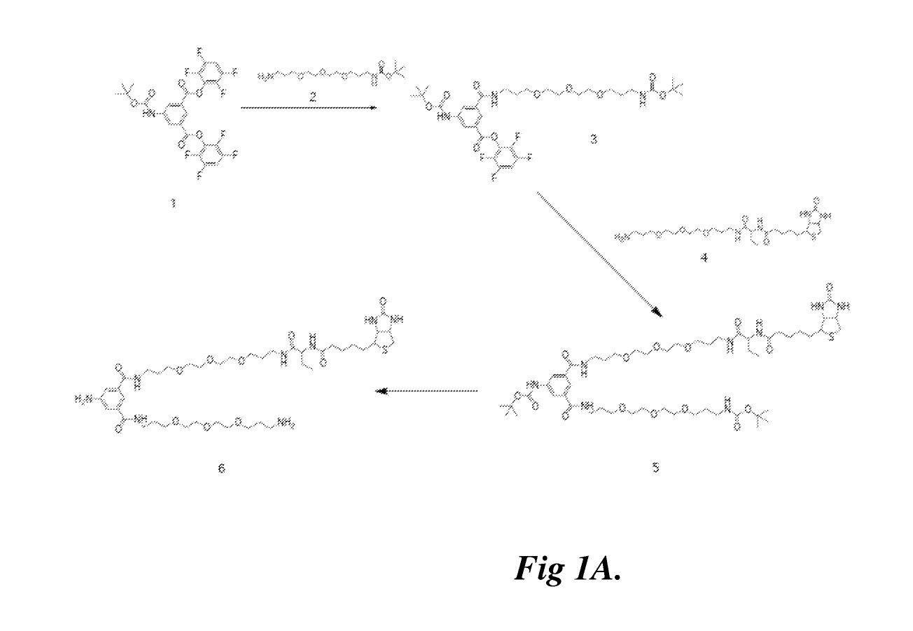

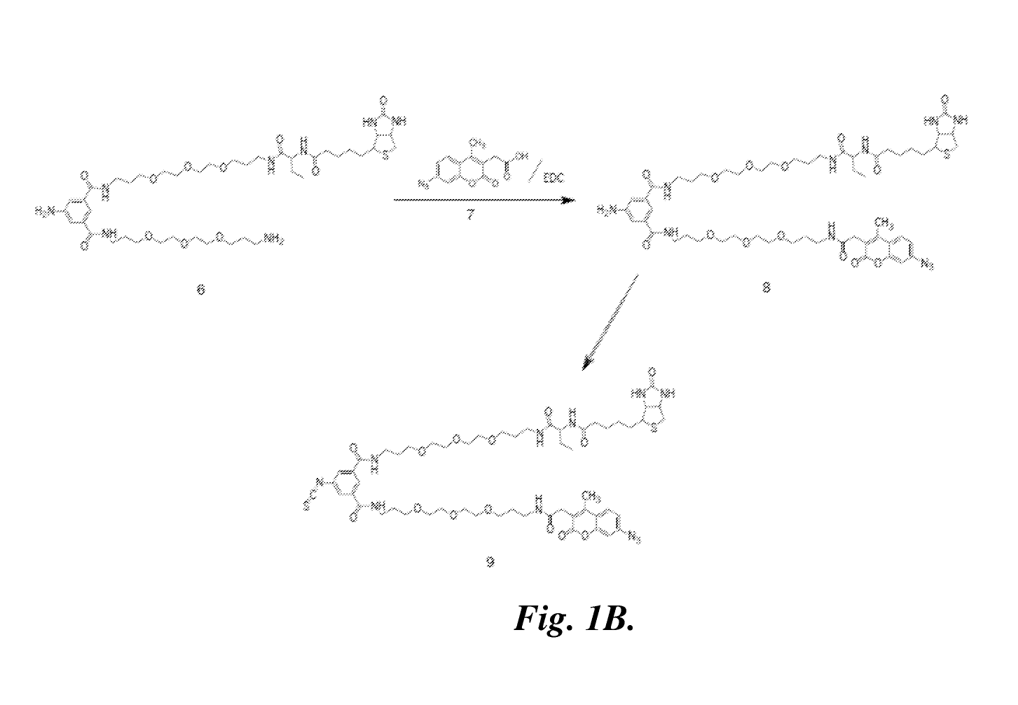

The invention also relates to a method to produce said macromolecule. Said method comprises the steps of; a) providing a branched polymer central core with at least one layer of monomers and comprising at least three functional groups; b) providing at least three linear polymers, wherein one polymer is longer than the other ones; and c) coupling said at least three linear polymers to said branched polymer central core to obtain said macromolecule. Examples on how to produce macromolecule are outlined in FIGS. 1 to 19 and in the examples.

The above defined macromolecule/macromolecule conjugate/macromolecule biotin conjugate kit or system may be used for the treatment or diagnosis of a mammal, such as a human being, in need thereof.

The invention also relates to a method of treating a mammal such as a human being suffering from a disease by administering a therapeutically effective amount of said macromolecule/macromolecule conjugate/macromolecule biotin conjugate. The mammal may be a patient being treated or analysed in any kind of therapeutic, diagnostic, research development or other applications.

Following examples are intended to illustrate, but not to limit, the invention in any manner, shape, or form, either explicitly or implicitly.

### EXAMPLES

In the experimentals described below the following abbreviation applies: TFA, refers to trifluoro acetic acid, DMF, refers to dimethyl formamide, HRMS (ES⁺) refers to electrospray high-resolution mass spectrometry, LRMS, refers to low resolution mass spectrometry, EDC, refers to 1-ethyl-3-(3-dimethylaminopropyl) carbodiimide, HOBt, refers to hydroxybenzotriazole, TCDI, refers to Thiocarbonyl diimidazole, Boc=tBoc refers to tert-butyloxycarbonyl, ELSD refers to Evaporative Light Scattering Detector, MAL−, refers to a maleimidyl group, dPEG™, refers to discrete polyethylene glycol chains from Quanta BioDesign, Powell, Ohio, US and HPLC, refers to high resolution liquid chromatography from Alltech Assoc., Inc, Deerfield, US using a reverse phase Alltima C18 5μ column at a flow rate of 1 mL/min and with a gradient of MeOH and 0.1% acetic acid, SEC, refers to size exclusive chromatography, using a Waters SW300-column.

### Example 1

### Preparation of a Trifunctional Biotinylation Reagent (11)

The trifunctional biotinylation reagent (11) was prepared according to FIG. 1.

Compounds 1, 2, 3, 4 and 7 in FIG. 1 were synthesized according to reports in the literature, as known to anyone skilled in the art. TFA salt of 4 (1.59 g, 2.46 mmol) was added to a solution of TFP ester 3 (1.80 g, 2.46 mmol), triethylamine (0.86 mL, 6.15 mmol) and anhydrous DMF (40 mL), then the resultant solution was stirred at rt for 1 h. After the reaction solution was evaporated by rotavap evaporator under vacuum, the crude product was purified by silica gel column (2.5×30 cm) eluted with 30% MeOH/EtOAc to afford 2.48 g (92%) of 5 as a colorless solid. HRMS (ES⁺) calcd for C₅₂H₈₉N₈O₁₅S(M+H)⁺: 1097.6168. Found: 1097.6155. HPLC 13.1 min.

Compound 5 (2.2 g, 2.00 mmol) was dissolved in neat TFA (10 mL) and stirred at rt for 10 min. After volatile materials were evaporated by a stream of argon, the residue was washed with EtOAc (3×100 mL) to receive 6 as a light-brown tacky solid. Yield 2.22 (98%). HPLC 7.7 min.

Di-TFA salt of compound 6 (0.50 g, 0.44 mmol) was dissolved in a solution of MeOH (5 mL) and triethylamine (0.5 mL) and stirred at it for 5 min. The solution was evaporated to dryness by rotavap evaporator under vacuum, then the residue was redissolved in anhydrous DMF (8 mL). To this solution compound 7 (115 mg, 0.44 mmol), EDC (128 mg, 0.67 mmol) and HOBt (120 mg, 0.89 mmol) were added respectively, the resultant solution was stirred at it for 2 h. The reaction solution was evaporated by rotvap evaporator, and then the crude product was purified by silica gel column (1.5×25 cm) eluted with 40% MeOH/EtOAc to afford 8 as a colorless solid. Yield 0.40 g (79%). HRMS (ES⁺) calcd for C₅₄H₈₀N₁₁O₁₄S(M+H)⁺: 1138.5607. Found: 1138.5603. HPLC 12.0 min.

TCDI (22 mg, 123.4 mmol) was added to a solution of compound 8 (100 mg, 87.8 mmol) and anhydrous DMF (4 mL), then the resultant solution was stirred at it for 1 h. The reaction solution was triturated with diethyl ether (15 mL). After solvents were decanted, the remained residue was washed with diethyl ether (3×15 mL), dried under vacuum for 2 h to give 98 mg (95%) of 9 as a yellow tacky solid. This compound was too unstable to get a good mass spectrum. HPLC 13.8 min.

A solution containing compound 9 (80 mg, 67.8 mmol), H2N-dPEG24-CO2H (80 mg, 69.8 mmol), triethylamine (20 mL, 143 mmol) and anhydrous DMF (4 mL) was stirred at rt for 1 h. The solution was evaporated by rotvap evaporator under vacuum, and then the residue was washed with EtOAc (3×15 mL), dried under vacuum for overnight to afford 10 as a light-yellow solid. Yield 142 mg (90%). HRMS (ES⁺) calcd for C₁₀₆H₁₇₈N₁₂Na₃O₄₀S₂ (M−2H+3Na)⁺: 2392.1398. Found: 2392.1442. HPLC 12.3 min.

TFAOTFP (10 mg, 38.2 mmol) was added to a solution of compound 10 (50 mg, 21.5 mmol), triethylamine (6 mL, 43 mmol) and anhydrous DMF (4 mL) at rt, then the resultant solution was stirred at rt for 10 min. After the solution was washed with 10% EtOAc/hexanes (3×15 mL) to provide 11 as a residue.

### Example 2

### Conjugation of the Linker (11) from Example 1 with a Dendrimer

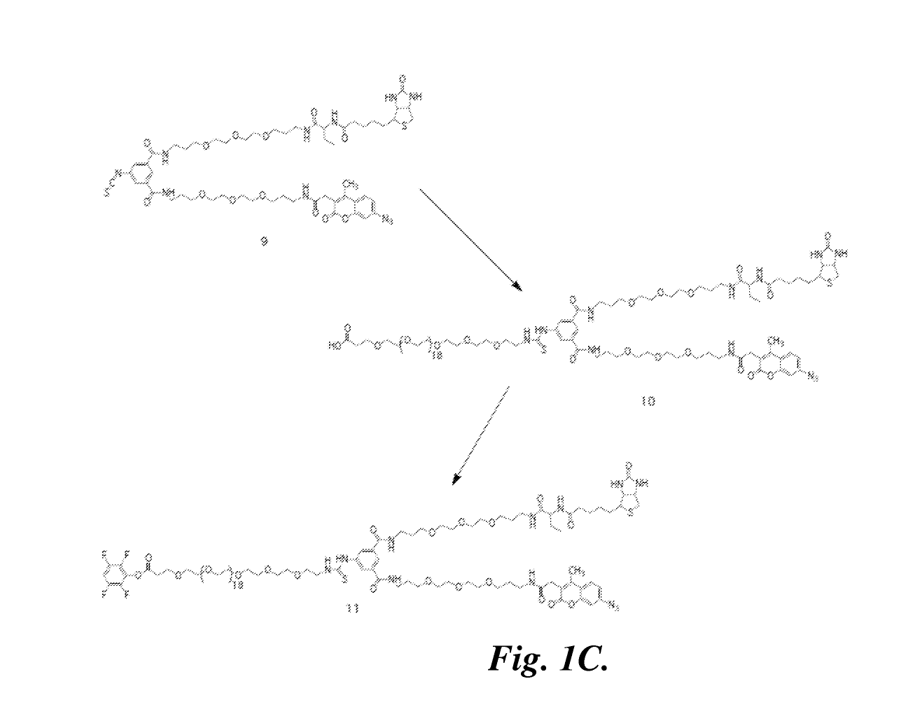

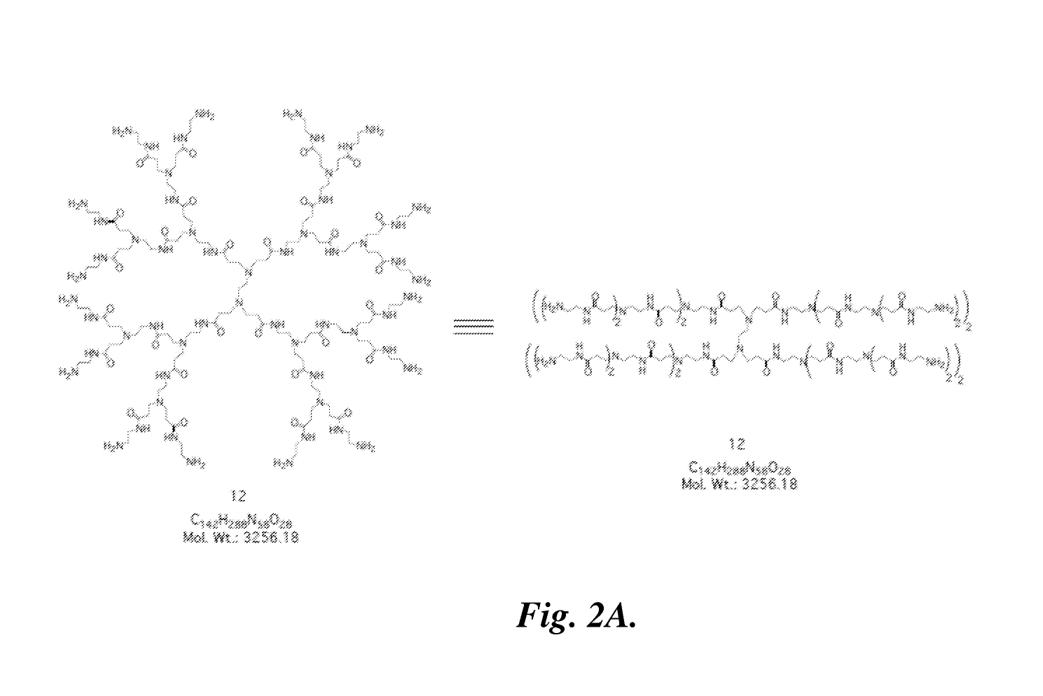

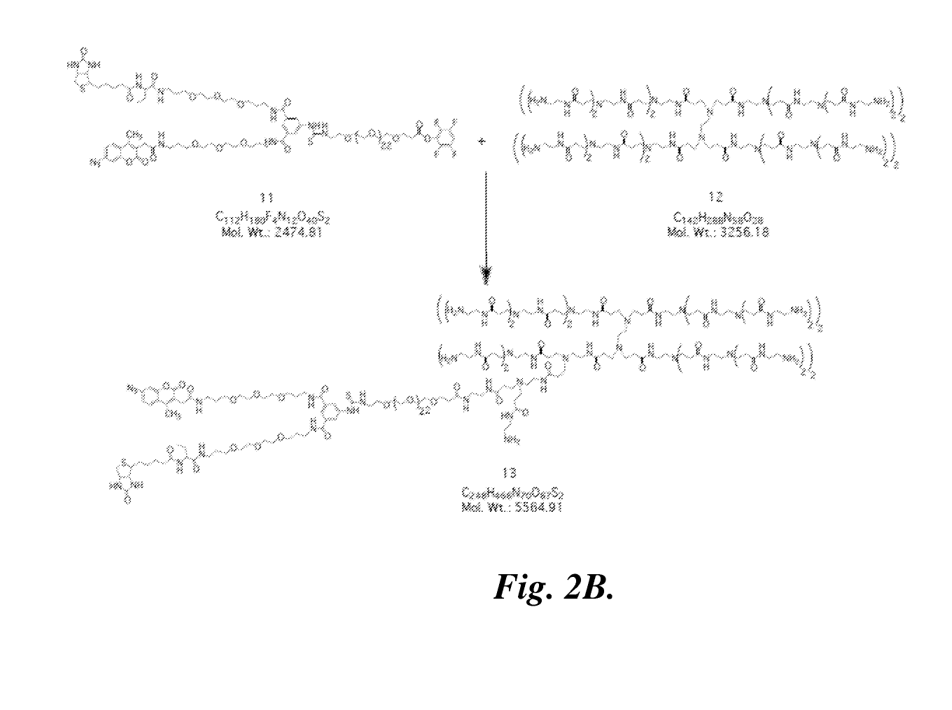

A dendrimer with one extended polymer (13) was prepared according to FIG. 2

The residue with 11 from Example 1 was redissolved in anhydrous DMF (4 mL) and added dropwise over 10 min to a solution of a 12, which is a generation 2 of polyamidoamino (PAMAM) dendrimer (Tomalia et al., (1990). Angew Chem Int Ed 29, 138-175, Tomalia et al., (2004). Aldrichimica Acta 37, 39-57). (0.60 g, 184 mmol), triethylamine (6 mL, 43 mmol) and anhydrous DMF (3 mL). The solution was stirred at rt for another 10 min. After the solution was evaporated under vacuum the residue was redissolved in water (3 mL) and purified by Sephadex G-25 column (2.5×45 cm) eluted with water to give 68 mg (57%) of 13 as a light-yellow tacky solid. LRMS (MALDI-TOF⁺) calcd for C₂₄₈H₄₇₀N₇₀O₆₇S₂ (M+4H)⁺: 5565.50. Found: 5565.51. HPLC 1.7 min.

### Example 3

### Synthesis of a Substituted Dendrimer that has a Biotin, an Aryl Amine and Several Discrete PEG (dPEG) Linkers with Terminal Protected Primary Amines

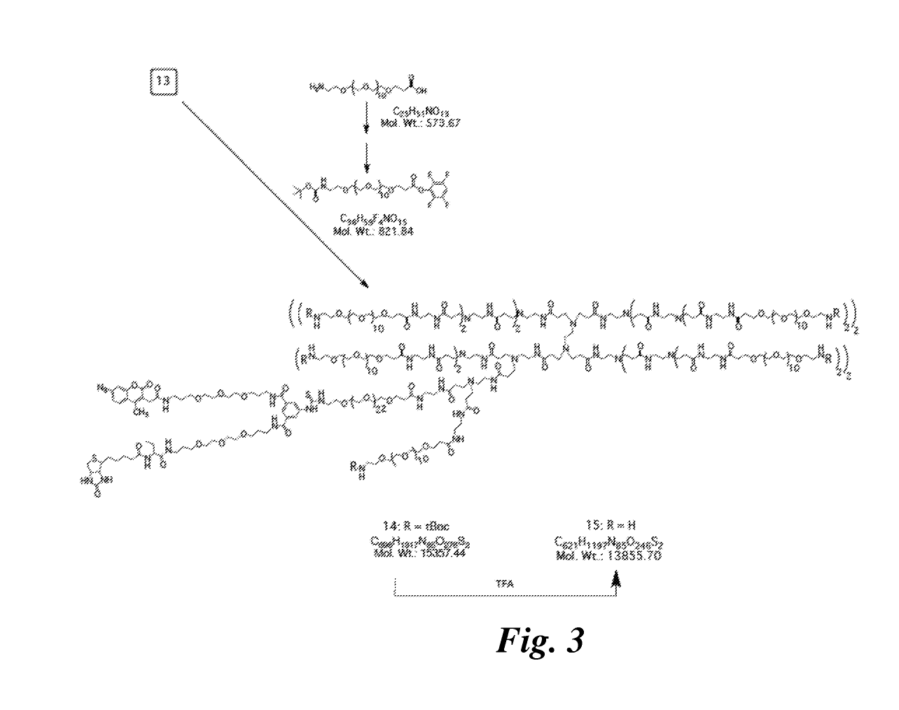

The protected macromolecule 14 was prepared according to FIG. 3.

BOC-dPEG™₁₂-CO₂-TFP (196 mg, 0.367 mmol), synthesized as described below in example 4, was added to a solution of compound 13 (68 mg, 0.012 mmol), triethylamine (68 mL, 0.489 mmol) and anhydrous DMF (5 mL), then the reaction solution was stirred at rt for 5 days. After solvents were evaporated by rotavap evaporator under vacuum, the residue was redissolved in water (3 mL) and purified by Sephadex G-25 column (2.5×45 cm) eluted with water to give 14 as a colorless tacky solid. Yield 184 mg (94%). HPLC 11.1 min.

### Example 4

### Synthesis of BOC-dPEG™₁₂-CO₂-TFP

(BOC)₂O (0.46 g, 2.10 mmol) was added to a solution of H₂N-dPEG™₁₂-CO₂H (1.0 g, 1.62 mmol), triethylamine (0.34 mL, 2.43 mmol) and anhydrous DMF (30 mL), then the resultant solution was stirred at rt for 1 h. After solvents were evaporated under vacuum, the residue was washed with 10% EtOAc/hexanes (3×30 mL), dried under vacuum for overnight to give BOC-dPEG™₁₂-CO₂H as a colorless oil product. Yield 1.12 g (96%). HRMS (ES⁺) calcd for C₃₂H₆₃NNaO₁₆(M+Na)⁺: 740.4045. Found: 740.4029. HPLC 11.0 min.

TFAOTFP (0.47 g, 1.79 mmol) was added slowly to a solution of BOC-dPEG™₁₂-CO₂H (1.0 g, 1.39 mmol), triethylamine (0.39 mL, 2.79 mmol) and anhydrous DMF (10 mL) under ice-bath temperature, then the resultant solution was stirred at the same temperature for 30 min. The solution was evaporated by rotvap evaporator under vacuum, then the residue was washed with 5% EtOAc/hexanes (3×20 mL), dried under vacuum for 4 h to afford BOC-dPEG™₁₂-CO₂-TFP as a colorless oil product. Yield 1.02 g (86%). HRMS (ES⁺) calcd for C₃₈H₆₃F₄NNaO₁₆ (M+Na)⁺: 888.3981. Found: 888.3991. HPLC 13.6 min.

### Example 5

### Deprotection of 14 and Insertion of a Linker II

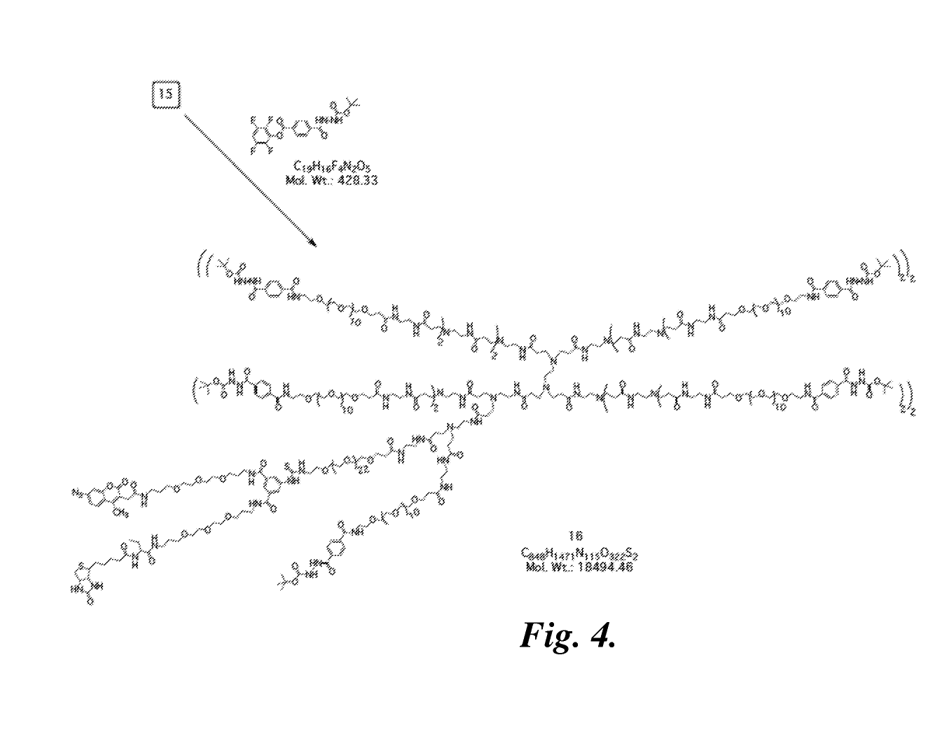

A substituted dendrimer that has a biotin, an aryl amine and several discrete PEG (dPEG) linkers with terminal protected acyl hydrazines was prepared according to FIG. 3 och FIG. 4.

Compound 14 (20 mg, 1.2 mmol) and neat TFA (1 mL) were stirred at rt for 20 min, then the excess TFA was removed by a stream of argon. The remained residue was washed with EtOAc (3×5 mL) to provide 15 as a residue. The residue was redissolved in anhydrous DMF (2 mL). BOC-NHNHCO-Ph-CO₂-TFP (16 mg, 37.4 mmol) and triethylamine (30 mL) were added respectively. After the reaction solution was stirred at rt for 4 days, the solvents were evaporated by rotavap evaporator, and the crude compound was purified by Sephadex G-25 column (2.5×45 cm) eluted with water to give 16 as a colorless tacky solid. Yield 23 mg (˜100%). HPLC 10.5 min.

### Example 6

### Removal of tBoc Protecting Groups to Provide Free Terminal Amines and Conjugation of Daunorubicin with the Deprotected Dendrimer 16 from Example 5

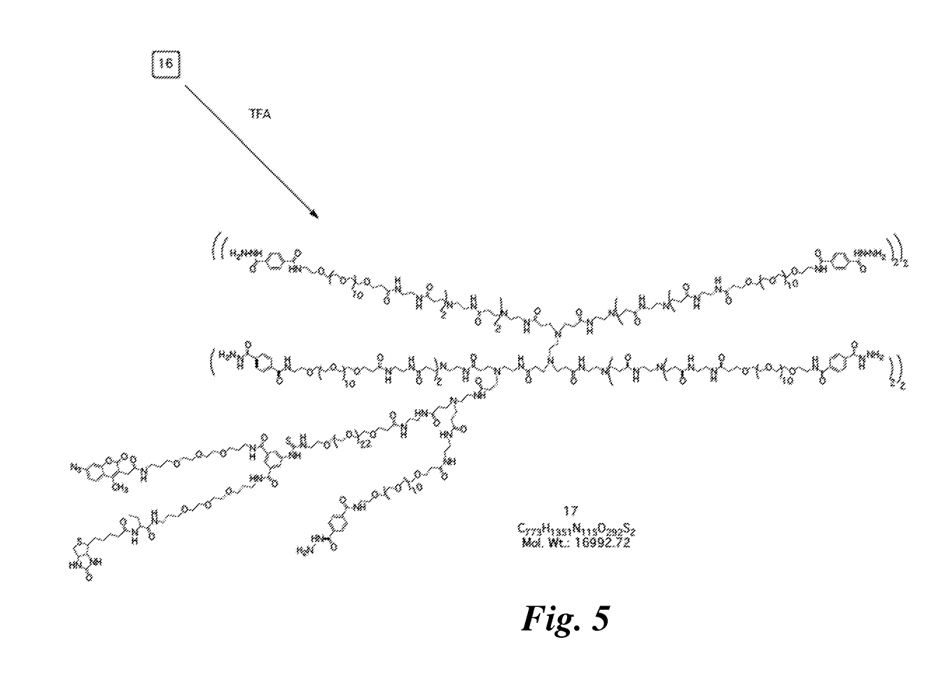

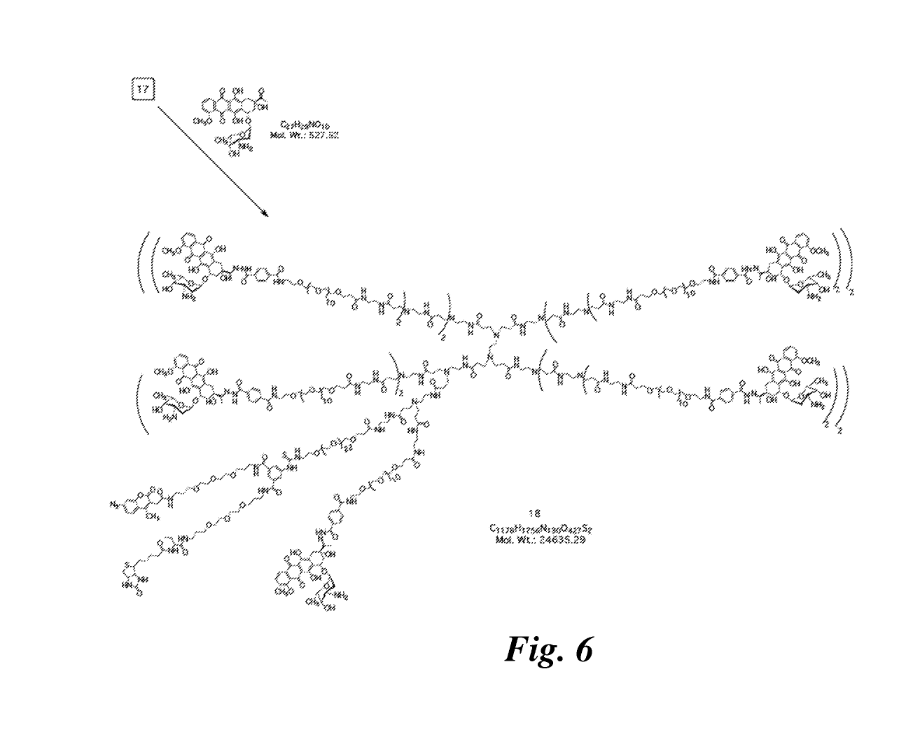

The dendrimer 18 caped with Daunorubicin was prepared according to FIGS. 5 and 6.

Compound 16 (15 mg, 0.81 mmol) and neat TFA (1 mL) were stirred at rt for 20 min, then the excess TFA was removed by a stream of argon to provide 17. Daunorubicin HCl (9 mg, 16.0 mmol), MeOH (1 mL) and EtOH (1 mL) were added respectively after the residue was put under vacuum for 2 h. The resultant solution was stirred and heated at 80° C. for 4 h. After the solvents were evaporated by rotavap evaporator, the crude compound was purified by Sephadex G-25 column (2.5×45 cm) eluted with water to give 18 as an orange tacky solid. Yield 18 mg (90%). HPLC 9.8 min.

### Example 7

### Insertion of a Maleimide in the Dendrimer Caped with Daunorubicin

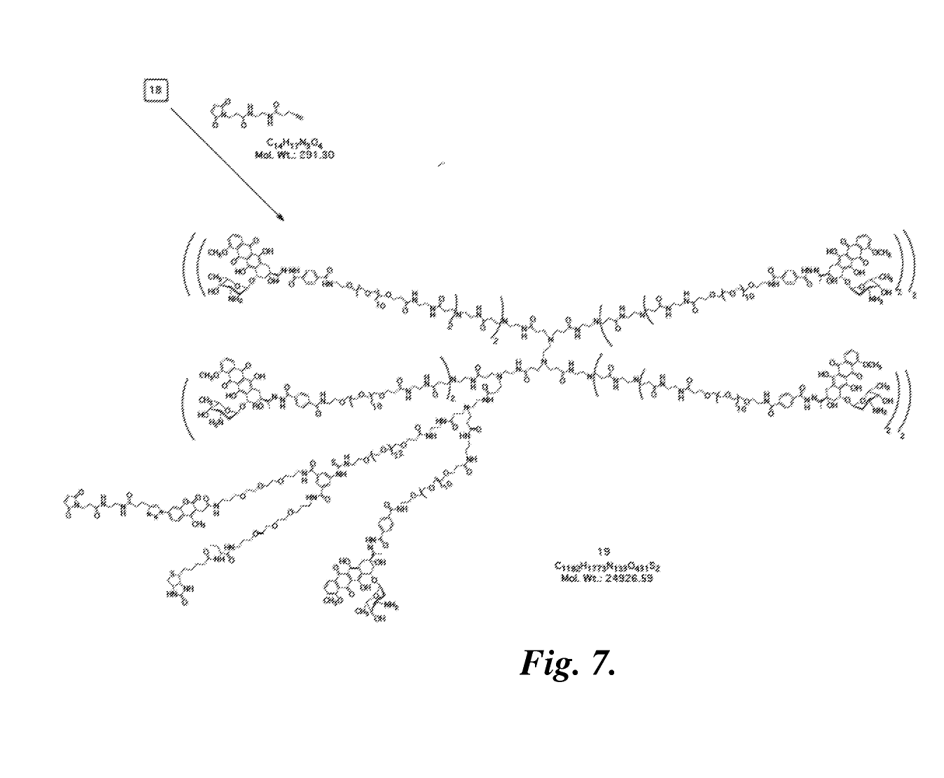

The dendrimer caped with Daunorubicin and with a maleimide group reactive towards sulfhydryls in antibodies was prepared according to FIG. 7

A 10 mg sample of 18 (0.405 mmol) was dissolved in 2 mL water. To this 130 μg Alkyne-maleimide (see FIG. 7, 0.446 mmol) in 130 μL DMF was added, followed by 32 μL 1M sodium ascorbate and 320 μL 100 mM copper sulfate. This was allowed to react for 2 hr. The DMF, water, and excess maleimide-alkyne were then removed by adding 10 mL of EtOAc, stirring for 10 min and removing the top layer. This was repeated 3 times, leaving 19 as a brown-orange residue.

### Example 8

### Synthesis of tBoc Protected Dendrimer-Coumarin-Azide with Trastuzumab

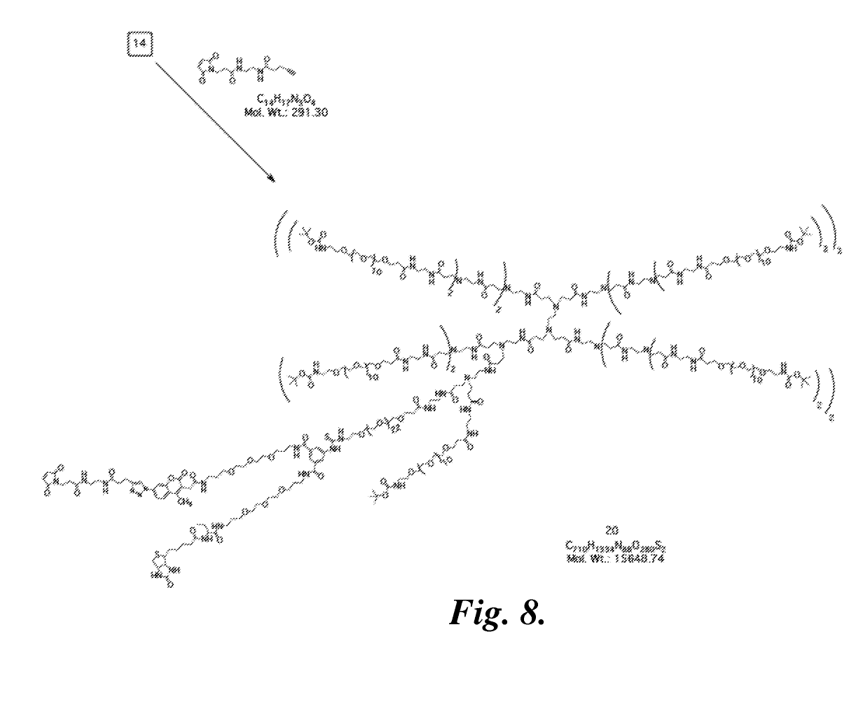

The maleimide bearing BOC-protected dendrimer 20 was prepared according to FIG. 8 and subsequently coupled to Trastuzumab as described below.

To 20 mg of 14 from example 3 (1.2 μmol) in 1 mL water was added 1.15 mg maleimide-alkyne (5.1 μmol) in 1 mL DMF, followed by 32 μL 1M Sodium ascorbate and 320 μL 100 mM Copper sulfate. This was allowed to react for 3 hr. The DMF, water, and excess maleimide-alkyne were then removed by adding 10 mL of EtOAc, stirring for 10 min and removing the top layer. This was repeated 3 times. The remaining residue was dried under a stream of air and re-dissolved in 916 μL of 1:1 DMF:water. This sample comprising 20 was use directly in the conjugation step.

To 8 mg of 10 mg/mL Trastuzumab in PBS was added 160 μL of 100 mM Dithiothreitol. After 1 hr at rt, the mixture was run over a PD-10 column (Sephadex G-25) that had been pre-equilibrated with PBS at pH 6.5 with 1 mM EDTA. The protein fractions were combined to yield 2.5 mL of 3.1 mg/mL DTT treated Trastuzumab. This was stored on ice until needed (30 min).

An 84 μL, aliquot of 20 was added 268 μL of 50 mM EDTA added (to chelate the copper). This was then added to 4 mg of DTT treated Trastuzumab. After 1 hr of gentle tumbling at rt, the mixture was run over a PD-10 column (Sephadex G-25) that had been pre-equilibrated with PBS at pH 7.0. The protein fractions were combined to give 2 mL at 1.7 mg/mL of 20 coupled to Trastuzumab. HABA analysis shows 2.5 biotins/antibody.

### Example 9

### Synthesis of a Dendrimer with dPEG™₂₄ and Terminal Protected Aryl Acyl Hydrazines

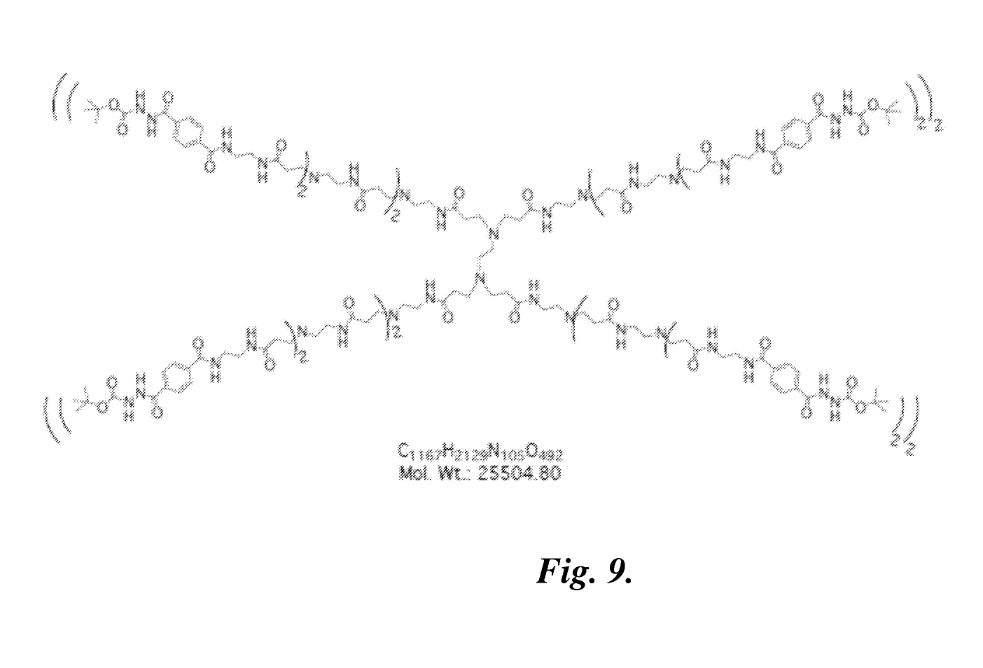

A symmetrical dendrimer with dPEG™₂₄ and terminal protected aryl acyl hydrazines 21 as shown in FIG. 9 was prepared as described below.

TFAOTFP (73 mg, 0.28 mmol) was added slowly to a solution of BOC-NHNHCO-Ph-CONH-dPEG™₂₄-CO₂H (300 mg, 0.22 mmol), triethylamine (45 mL, 0.32 mmol) and anhydrous CH₃CN (15 mL) under ice-bath temperature, then the resultant solution was stirred at the same temperature for 20 min. The solution was evaporated by rotvap evaporator under vacuum, then the residue was washed with 10% EtOAc/hexanes (3×20 mL), dried under vacuum for 4 h to afford BOC-dPEG™₂₄-CO₂-TFP as a colorless oil product. Yield 319 mg (96%). HPLC 12.9 min.

A solution of Dendrimer generation 2 of polyamidoamino (PAMAM) dendrimer, see 12 in FIG. 2, (Tomalia et al., (1990). Angew Chem Int Ed 29, 138-175, Tomalia et al., (2004). Aidrichimica Acta 37, 39-57). (20 mg, 6.14 mmol), BOC-dPEG™₂₄-CO₂-TFP (250 mg, 160.6 mmol), triethylamine (30 mL, 215.24 mmol) and anhydrous DMSO (4 mL) was stirred at rt for 20 h. The solution was triturated with 10% EtOAc/hexanes (20 mL), then the solvents were decanted and the residue was washed with 20% EtOAc/hexanes (3×20 mL). The crude residue was redissolved in water (4 mL) and was purified by Sephadex G-25 column (2.5×45 cm) eluted with water to give 21 as a colorless tacky solid. Yield 142 mg (91%). HPLC 10.7 min.

### Example 10

### Deprotection of a Dendrimer with dPEG™₂₄ and Terminal Protected Aryl Acyl Hydrazines and Coupling to Daunorubicin

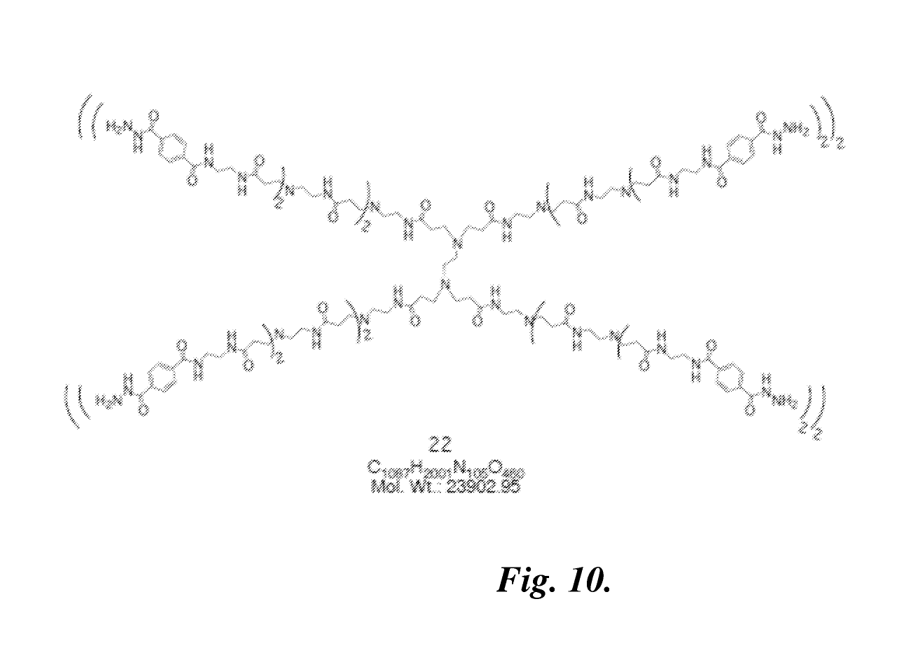

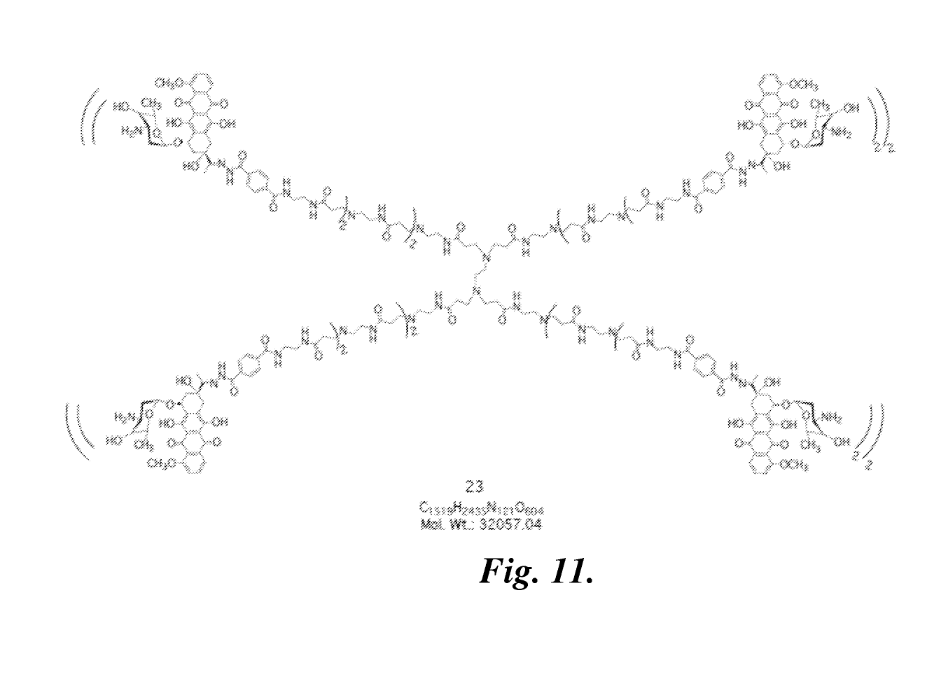

A symmetrical dendrimer with dPEG™₂₄ and terminal aryl acyl hydrazines 22 as shown in FIG. 10 was prepared as described below and subsequently coupled to Daunorubicin, as described below, to afford the symmetrical Daunorubicin capped dendrimer 23 as shown in FIG. 11.

Compound 21 (83 mg, 3.25 mmol) and neat TFA (4 mL) were stirred at rt for 20 min. After excess TFA was removed by a stream of argon, the residue was washed with EtOAc (3×15 mL), dried under vacuum for 2 h.

Then the TFA deprotected intermediate 22 was redissolved in MeOH (6 mL) and EtOH (4 mL), then Daunorubicin HCl (36.7 mg, 65.08 mmol) was added. The resultant solution was stirred and heated at 85° C. for 4 h. The solution was cooled to rt and the solvents were removed by rotvap evaporator under vacuum. The crude compound was redissolved in water (5 mL) and purified by Sephadex G-25 column (2.5×45 cm) eluted with water to give 23 as an orange tacky solid. Yield 56 mg (54%). HPLC 9.9 min.

### Example 11

### Synthesis of TPF-dPEG™₂₄-dPEG4-azide

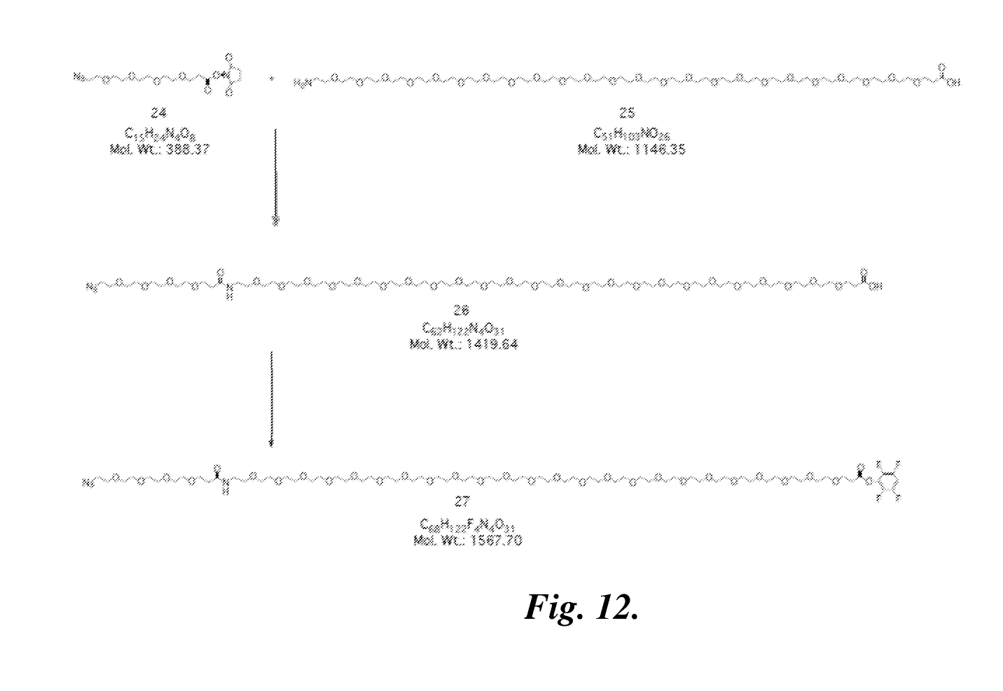

An example of an extended polymer (a), TPF-dPEG™₂₄-dPEG4-azide (27), was prepared according FIG. 12.

To a solution of 0.5 g dPEG™₂₄ (25) (0.44 mmol) in 1 mL DMF was added a solution of 204.2 mg dPEG™₄-azide (24) (0.44 mmol) in 1 mL DMF. After stirring for 15 min, the mixture was concentrated to 1 mL and transferred into 12.5 mL ether to precipitate the product. This yielded 427.5 mg (81%) of 26.

To 150 μL of TFP-TFA in 2 mL Acetonitrile at 0° C. was added 102 mg of 26 (0.072 mmol) and 200 μL triethylamine. The reaction was allowed to proceed for 12 min. The product was precipitated by addition of 10% ether in hexanes and washed with 2×10 mL of that solvent. This yielded 15 mg of 27.

### Example 12

### Synthesis of Dendrimer with One Extended Polymer, which has an a Terminal Azide

A dendrimer (28) with one extended polymer, which polymer has a terminal azide, was prepared according to FIG. 13.

42.9 mg (27.4 mmol) 27 from example 11 and 267.3 mg (82.1 mmol) generation 2 of polyamidoamino (PAMAM) dendrimer, see 12 in FIG. 2, (Tomalia et al., (1990). Angew Chem Int Ed 29, 138-175, Tomalia et al., (2004). Aldrichimica Acta 37, 39-57) were dissolved in 20 mL DMF and stirred with 124 μL (82.1 mmol) triethylamine for 1 hr. The mixture was concentrated under vacuum. The residue was dissolved in 2 mL water and purified by SEC. This gave 36 mg of pure 28.

### Example 13

### Synthesis of TFP-dPEG™₁₂-benzylketone

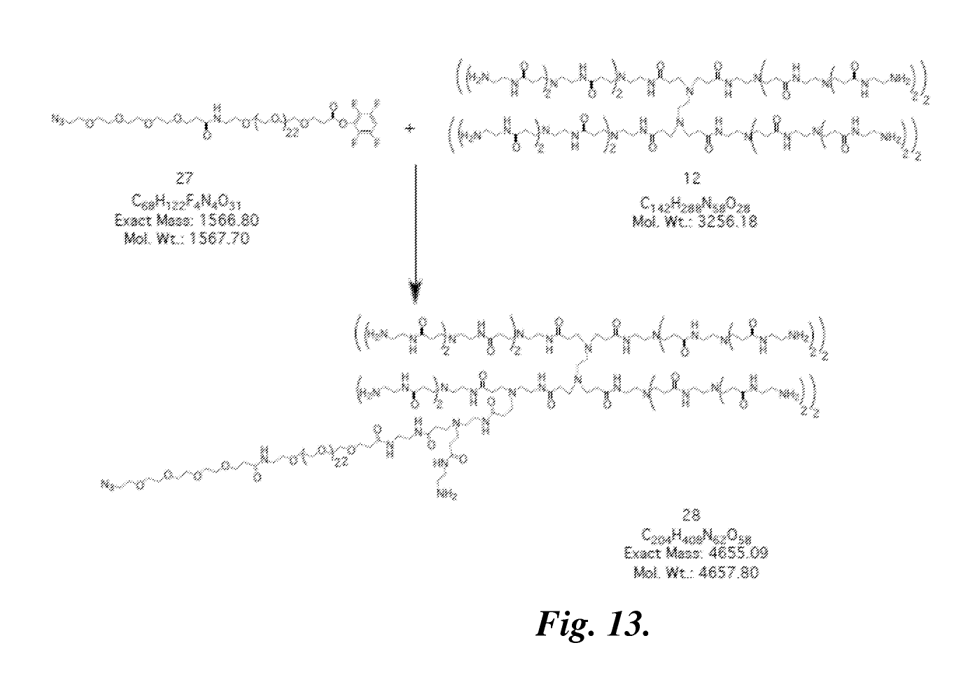

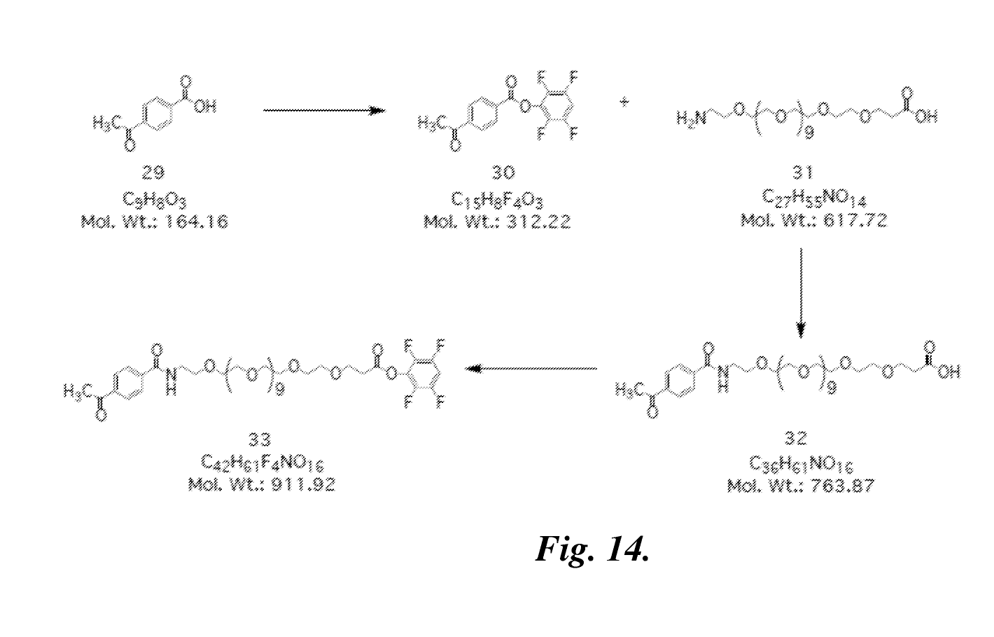

A linear polymer (33) with a terminal benzylketone was prepared according to FIG. 14 and as described blow.

1 g (6.1 mmol) 4-Acetylbenzoic acid (29) and 3.45 mL Triethylamine (4 eq) were dissolved in 10 mL DMF, followed the addition of 3.2 g of 75% pure TFP-TFA. HPLC showed that the reaction was complete after 5 min at rt. The mixture was diluted with 90 mL DI water and stirred for 5 min. The precipitate was collected by filtration and washed with DI water. This yielded 1.836 g (96.5%) of 30 as a gray-yellow solid of. MS+Na 335.0298; ELSD=13.89 min

1 g amino-dPEG™₁₂-acid (31, 1.62 mmol) and 458 μL Triethylamine (2 eq) were mixed in 10 mL DMF. To this suspension was added solid 30 causing the mixture to become clear. HPLC showed that the reaction was complete after 35 min at rt. The mixture was concentrated and washed with 1 mL EtOAc into a solution of 10:1 Hexanes:EtOAc. After vortexing for 1 min, the mixture was centrifuged and the top layer removed. This was repeated, and the bottom layer was transferred to a flask and diluted with 1 mL EtOAc and 20 mL Hexanes. This was stirred overnight and the resulting precipitate was recovered as a brown solid of 32. MS+Na 786.3867 ELSD=9.54 min

To a solution of 228 mg 32 (0.298 mmol) and 168 μL (4 eq) Triethylamine in 2 mL acetonitrile was added TFP-TFA at 0° C. HPLC showed that the reaction was complete after 7 minutes. The mixture was concentrated and dried in vacuo before dissolving in 0.5 mL EtOAc and transferring to a flask containing 10 mL of 1:9 ether:Hexanes. The top layer was removed and the bottom layer was treated with another 10 mL of ether:Hexanes. The bottom layer was concentrated to a viscous brown oil of 33. ELSD=12.63 min

### Example 14

### Synthesis of Dendrimer with a Terminal Azide and Several dPEG:s with Terminal Benzylketone

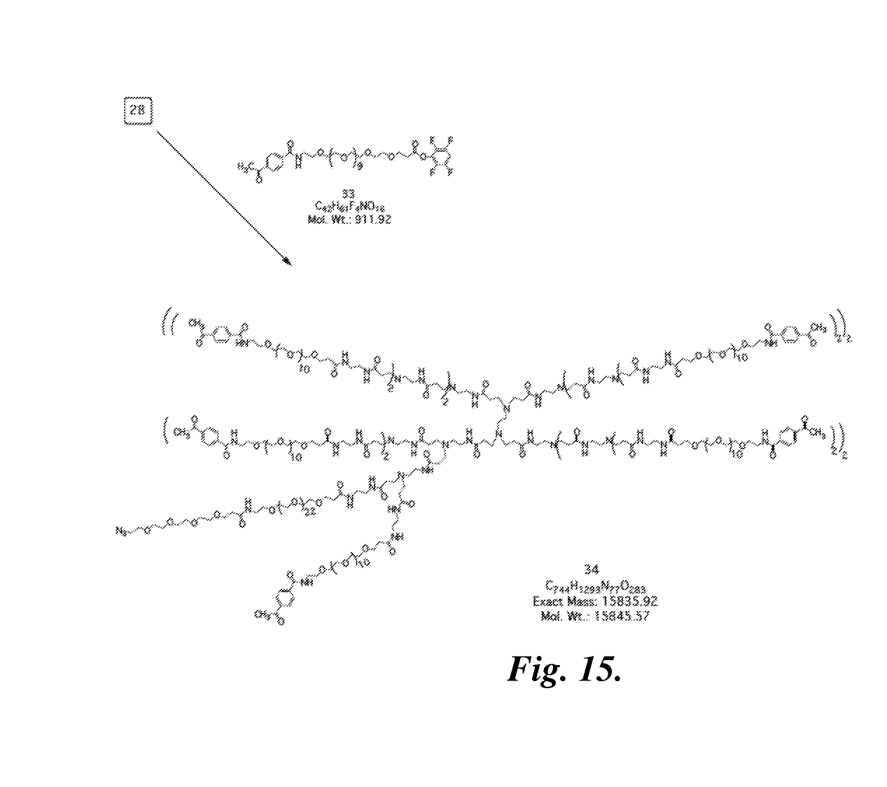

A dendrimer (34) with a terminal azide and several dPEG:s with terminal benzylketone was prepared according to FIG. 15 and as described blow.

36 mg dendrimer 28 and 528 mg (3 eq) TFP ester of 33 were dissolved in 3 mL DMF with 49 μL (3 eq) triethylamine. This was allowed to stir for 16 hr at rt. Half of the reaction was worked up at this time. The mixture was concentrated by vacuum and washed with 9 mL EtOAc:Ether 5:4 and 2 mL EtOAc sequentially. This gave 25 mg of 34 as a brown viscous oil.

### Example 15

### Synthesis of Dendrimer with a Terminal Azide, which Dendrimer is Capped with Desacetylvinblastine Hydrazide

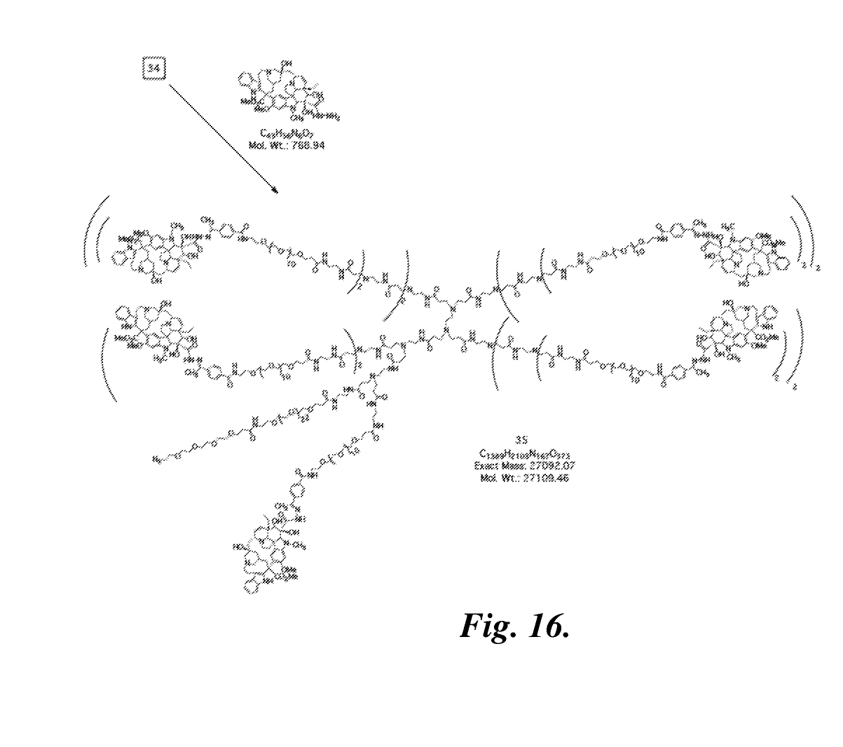

A dendrimer (35) with a terminal azide, which dendrimer is capped with Desacetylvinblastine was prepared according to FIG. 16 and as described blow.

36 mg Vinblastine-hydrazone, prepared from Vinblastine according to literature procedures (e.g. U.S. Pat. No. 4,203,898 and Helvetica Chimica Acta, 58 (6), 1690-1719, in the latter the synthesis of various hydrazine derivatives of vica alkaloids is described) was dissolved in methylene chloride to which 20 μL TFA was added. The mixture was concentrated and dried for 10 min under vacuum. The residue was mixed with 6.5 mL dry MeOH and heated to 36° C. for 5 min to facilitate dissolution. To this, a solution of 31 mg Keto-dendrimer 34 in 2.4 mL dry MeOH was added and stirred for 24 hr at rt. The mixture was concentrated and the residue was taken into 2 mL water and filtered. The filtrate was then purified by SEC to give 35.

### Example 16

### Synthesis of Dendrimer with a Terminal Maleimde, which Dendrimer is Capped with Desacetylvinblastine Hydrazide

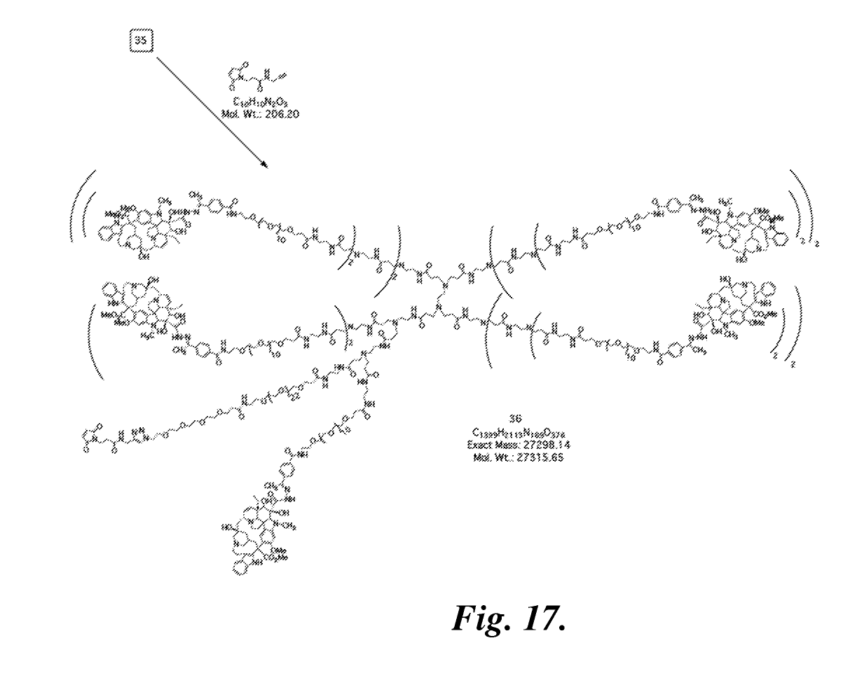

A dendrimer (36) with a terminal azide, which dendrimer is capped with Desacetylvinblastine was prepared according to FIG. 17 and as described blow.

Azido-Vinblastine-Dendrimer 35 (12 mg, 0.44 mmol) and Alkyne-maleimide (see FIG. 17 0.26 mg, 0.88 mmol) were dissolved in 0.4 mL of DMF/water (50/50), then CuSO₄ (14.1 mL, 0.088 mmol, 1 mg/mL in water) and ascorbic acid, Na salt (26.3 mL, 0.133 mmol, 1 mg/mL in water) were added respectively. The resultant solution was stirred at rt for 1 h, and then the crude product was purified by Sephadex G-25 column (2.5×45 cm) eluted with water to give 36 as a colorless tacky solid. Yield 11 mg (92%). HPLC 10.0 min.

### Example 17

### Synthesis of Dendrimer with a Terminal Isothiocyanate, which Dendrimer is Capped with Desacetylvinblastine Hydrazide

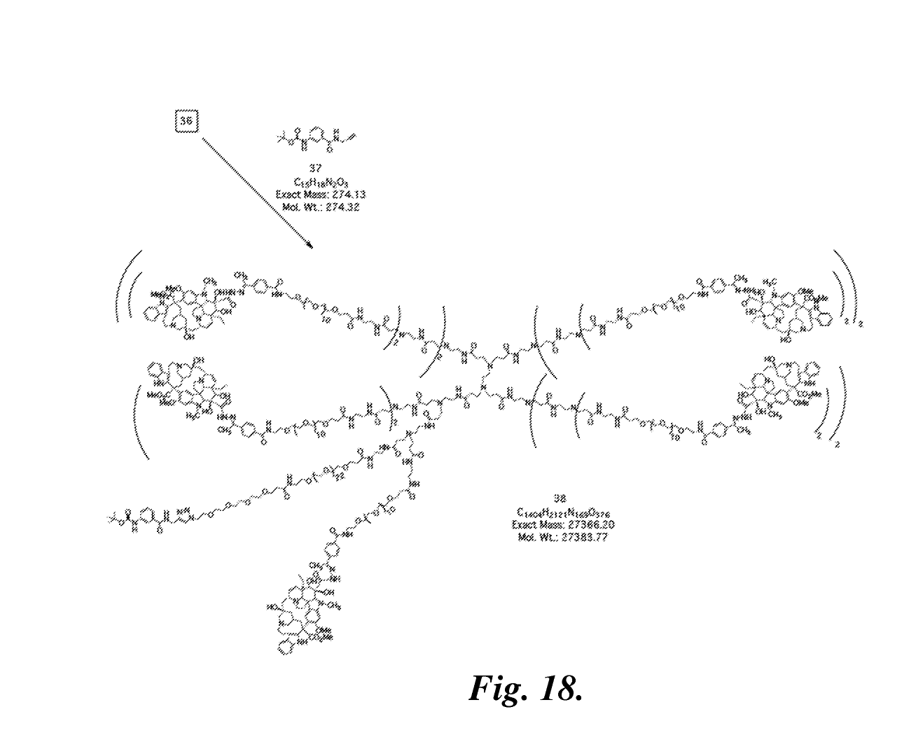

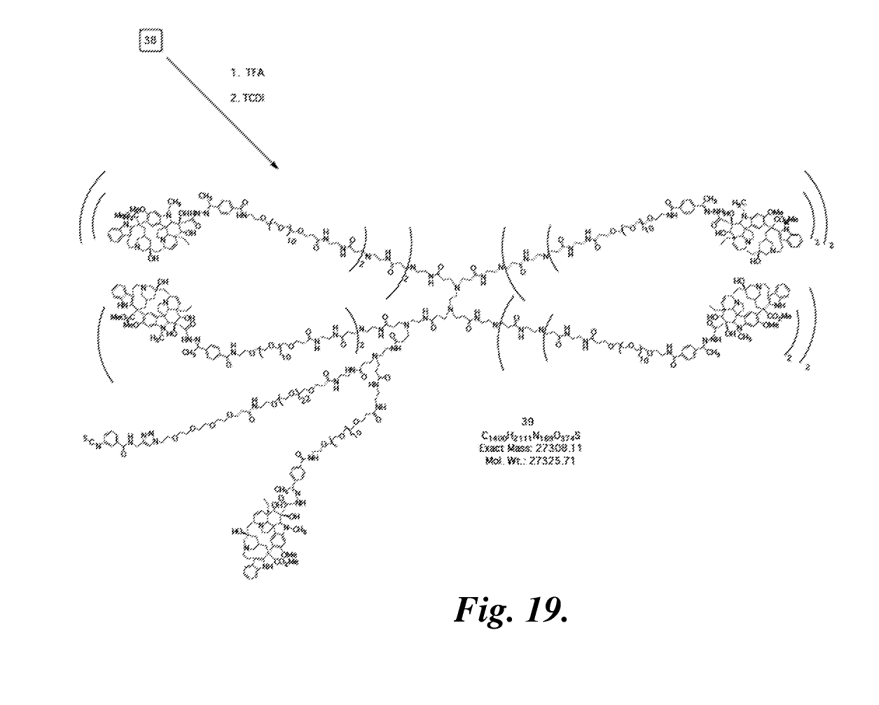

A dendrimer (38) with a terminal isothiocyanate, which dendrimer is capped with Desacetylvinblastine was prepared according to FIGS. 18 and 19 and as described blow.

Azido-Dendrimer 36 compound (12 mg, 0.44 mmol) and 37 (0.20 mg, 1.15 mmol) were dissolved in 0.4 mL of DMF/water (50/50), then CuSO₄ (14.1 mL, 0.088 mmol, 1 mg/mL in water) and ascorbic acid, Na salt (26.3 mL, 0.133 mmol, 1 mg/mL in water) were added respectively. The DMF, water, and excess 37 were then removed by adding 10 mL of EtOAc, stirring for 10 min and removing the top layer. This was repeated 3 times, leaving 19 as a brown-orange residue. The brown-residue comprising 19 was deprotected as described in example 5. The residue was dissolved in EtOAc and the solution was stirred at rt for 1 h, TCDI (1 mg, 5.61 mmol) was added and stirred at rt for another 1 h. The reaction solution was washed with EtOAc (5×5 mL), dried under vacuum for 2 h to give 39 light-yellow tacky solid. Yield 12 mg (˜100%). HPLC 9.9 min.

### Example 18

### Characterization of the Isothiocynate Derivative with Desacetylvinblastine Hydrazide (39)

Compound 39 was subjected to size-exclusion chromatography using a Waters Protein-Pac SW 300 column with 10% DMF in 20 mM sodium phosphate buffer, pH 7.0. The retention time of the major peak was determined to 9.2 min whereas free Vinblastine was eluted at 13.2 min. After incubation at pH 7.0 for 28 hrs at 37° C. no free desacetylvinblastine hydrazide was detected. However, incubation at pH 3.0 yielded an immediate release of material corresponding to the retention time of Vinblastine.

A UV spectra of the released material corresponds to that of Compound 39 and Vinblastine. UV scanning of the peak denoted Compound 39 as well as the peak corresponding to the retention time of Vinblastine did not indicate heterogeneous material.

### Example 19

### Analyses of the Affinity of the Binding to the Target Antigen (Immunoreactivity)

The influence of the conjugation process on the binding affinity (strength) of trastuzumab to the target antigen was studied utilizing a competitive inhibition assay.

Briefly, increasing amounts of Trastuzumab and 20 coupled to Trastuzumab (see example 8) were mixed with a constant amount of ¹¹¹In-labelled 1033-Trastuzumab. The mixtures were added to fixed SK-BR3 cells in 96 plate wells. After incubation for 2 hours at room temperature, the wells were washed, and the radioactivity bound to the cells was measured in an automatic NaI(Tl) scintillation well counter.

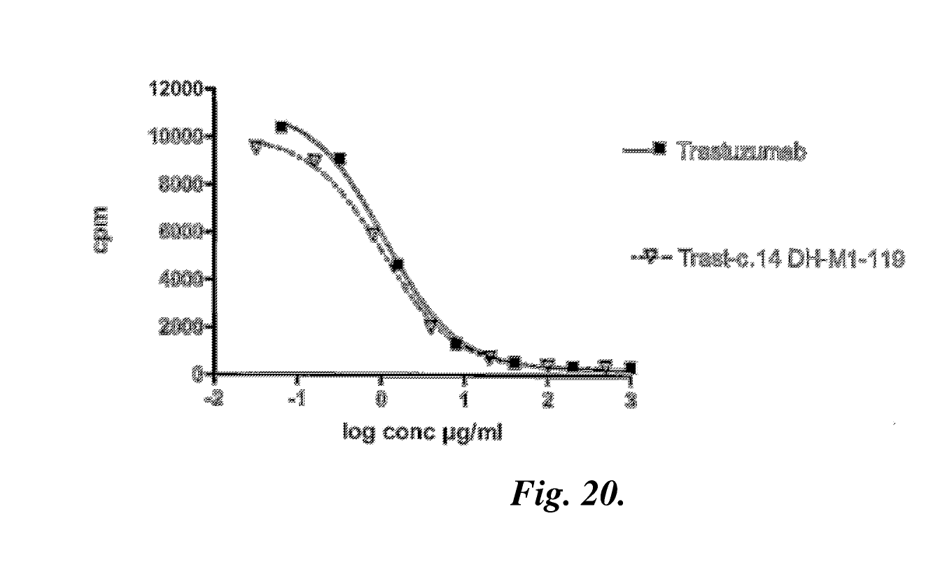

The amount of bound radioactivity was plotted against the concentration of trastuzumab and c14-trastuzumab (FIG. 20), and the concentration required for 50% inhibition (IC₅₀) was calculated. The IC₅₀ is a measure of the relative affinity (avidity) of the tested antibody; a decrease of affinity is seen as an increased IC₅₀ concentration.

1 μg/ml (6.7 nM) of ¹¹¹In-1033-Trastuzumab is inhibited by 0.03-500 μg/ml cold non-conjugated trastuzumab and 20 conjugated to Trastuzumab, respectively. The IC₅₀ was determined to 1.11 μg/ml (2.5 nM) and 1.06 μg/ml for 20 conjugated to Trastuzumab. Therefore, it was concluded that conjugation of trastuzumab with an average of up to 2.04 dendrimers per antibody would not diminish the binding properties of the antibody.

### Example 20

### Pharmacokinetics of 20 Conjugated to Trastuzumab

The pharmacokinetics in blood of 20 conjugated to trastuzumab is compared to the data obtained with non-conjugated trastuzumab.

Six (6) rats of the Brown Norwegian (BN) strain were injected intravenously with approximately 100 μg/rat of dendrimer-c14 conjugated trastuzumab and non-conjugated trastuzumab, respectively. About 0.2 ml blood was obtained from the tail vein on following occasions: 10 min, 1, 6, 24, 48 and 96 hours after injection. The concentration of trastuzumab was measured by an ELISA assay and expressed in per cent of injected activity per ml plasma.

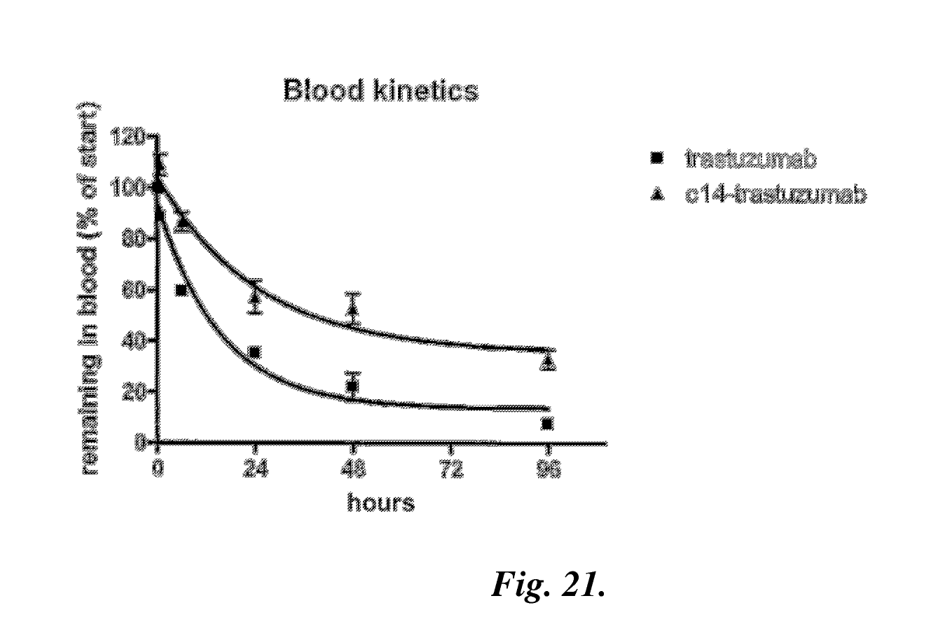

When blood clearance of 20 conjugated to Trastuzumab was compared to that of trastuzumab, a slightly prolonged half life in blood was seen (FIG. 21). This is expected for proteins conjugated with PEG.

### Example 21

### Size Distribution of 20 Conjugated to Trastuzumab

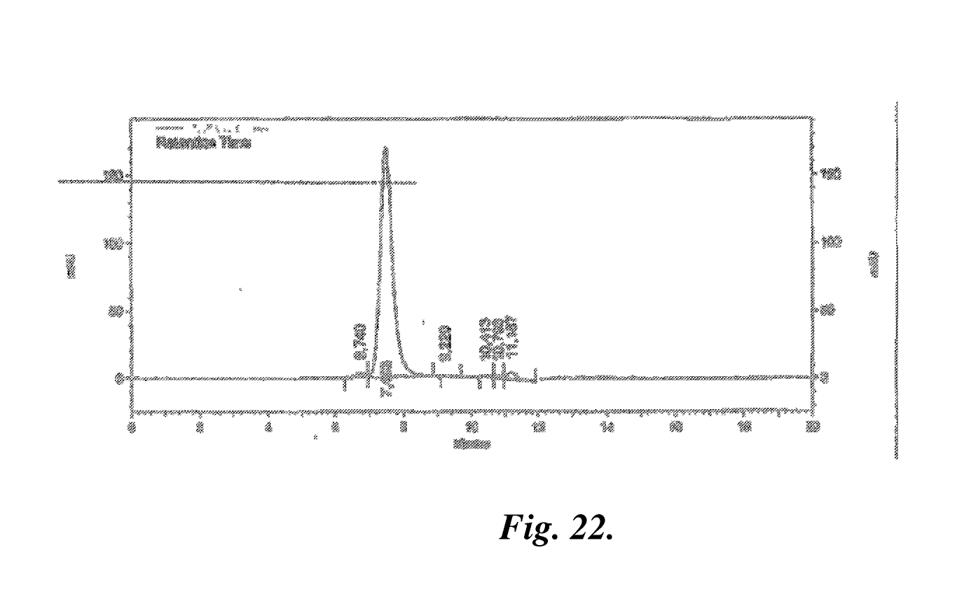

To determine the size distribution of 20 conjugated to trastuzumab, HPLC size exclusion chromatography was performed on a BioSep S3000 column PEEK (300×7.5 mm) in a 100 mM NaH₂PO₄ buffer. 20 μl of a sample at 2 mg/ml was injected. The effluent was monitored at 280 nm for detection of protein and at 320 nm for detection of dendrimeric structure. The results were compared with a molecular weight reference curve and non-conjugated trastuzumab. (FIG. 22).

A small fraction of aggregates was detected (1.6%), and low levels of fragments (3.5%) were also seen. Monitoring at 320 nm for detection of the dendrimer structure indicated that 2.5% of this is free dendrimer. The majority (95%) was eluted as expected for a monomer antibody conjugate i.e. slightly larger than non-conjugated trastuzumab (retention time 0.2 minutes shorter).

### Example 22

### Analysis of Number 20 Conjugated to Trastuzumab

This assay is based on the binding of the dye HABA ((4′-HydroxyazoBenzene-2-Benzoic Acid) to Avidin and the ability of Biotin to displace the dye in stoichiometric proportions. This displacement of dye is accompanied by a change in absorbance at 500 nm.

When a sample of 20 conjugated to trastuzumab at a concentration of 1.87 mg IgG per ml (12.5 μM) was analysed the biotin concentration was determined to 25.4 μM. Therefore, the molecular ratio of biotin i.e. dendrimer per IgG per molecule of trastuzumab was 2.04.

### Example 23

### Release of Drug from Drug-Conjugated Dendrimer

The kinetics of release of daunomycin from a starburst generation 2 dendrimer with dPEG′₂₄ arms conjugated with daunomycin via a hydrazone linker (compound 23 in example 10) was analysed.

The daunomycin-dendrimer was incubation at pH 7.0 and pH 4.5, respectively. At different time of incubation (0, 1, 2, 4, 6, 13, 24, 70, and 120 hours) at 37° C. samples was drawn for analyses of the amount of free daunomycin. The concentration of free daunomycin was analysed by high performance liquid chromatography (HPLC). The reverse phase HPLC was conducted utilising a Chromolith C-18 column with gradient elution of 10-90% acetonitrile in 0.1 M Ammonium Acetate buffer pH 6.0. The daunomycin was quantified at 488 nm utilising a diode array detector.

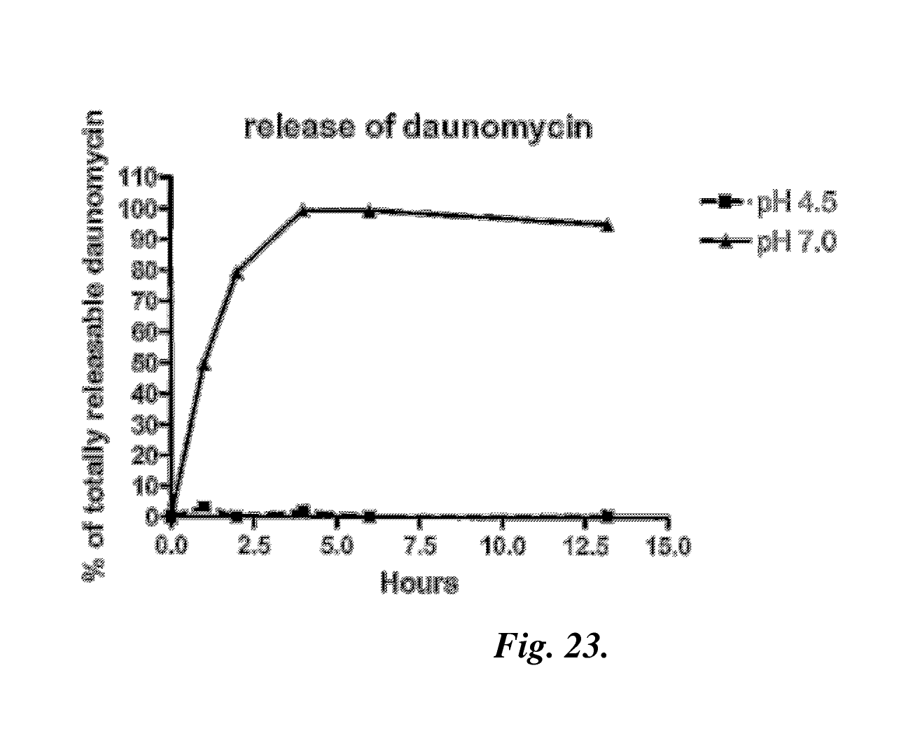

At pH 4.5, 70% was released within 6 hours, with no major release after that. At pH 7.0, no released daunomycin could be detected during the first 13 hours (FIG. 23).

1. A macromolecule comprising;
a. a polymer central core having at least two atoms to which at least two monomers are attached forming a dendrimeric structure comprising at least three functional groups,
b. at least two linear polymers (b) each being bound to one of said functional groups, wherein said polymer (b) at least have one terminal functional groups for cytotoxic agents, and
c. one extended polymer (a) being at least 1 atom longer than said polymer (b), being bound to one of said functional groups and having one terminal functional group for a targeting agent.

2-4. (canceled)

5. The macromolecule according to claim 1, wherein said dendrimeric structure is diaminobutanepoly(propylene imino) DAB or (polyamino amide) PAMAM.

6. The macromolecule according to claim 1, wherein said polymer (b) and (a) are selected from the group consisting of polyamino acid, dextran, polysaccharides, polypropylene oxide (PPO), a copolymer of polyethylene glycol (PEG) with PPO, PEG, polyglycolic acid, polyvinyl pyrrolidone, polylactic acid, and polyvinylalcohol.

7. The macromolecule according to claim 1, wherein said polymer (b) and (a) are discrete PEG (dPEG).

8-10. (canceled)

11. The macromolecule according to claim 7, wherein said polymer (b) comprises at least 8 —CH₂CH₂O— units.

12. The macromolecule according to claim 7, wherein said polymer (a) comprises at least 12 —CH₂CH₂O— units.

13. The macromolecule according to claim 1, wherein said polymer (b) comprises a linker II bound to said terminal functional group, wherein said linker II has a functional group capable of forming a degradable, biodegradable or releasable group with a functional group in said cytotoxic agent.

14. The functional group according to claim 13, wherein said functional group capable of forming a degradable, biodegradable or releasable group with a functional group in said cytotoxic agent, is an activated carbonate, an activated carbamate, a sulfhydryl or a hydrazide.

15. The macromolecule according to claim 1, further comprising a cytotoxic agent bonded, directly or via a linker II, to said terminal functional group on polymer (b).

16. The macromolecule according to claim 15, wherein the group which links the cytotoxic agent to the macromolecule is a carbonate, a thiocarbonate, a carbamate, a thiocarbamate, a urea, a thiourea, a disulfide or a hydrazone.

17. The macromolecule according to claim 15, wherein said cytotoxic agent is selected from the group consisting of taxanes, vinblastine, vincristine, desacetyl vinblastine, desacetyl vinblastine hydrazine, daunorubicin, geldanamycin, ricin, abrin, diphtheria toxin, modecin, tetanus toxin, mycotoxins, mellitin, α-amanitin, pokeweed antiviral protein, ribosome inhibiting proteins, auristatin E, auristatin EB (AEB), auristatin EFP (AEFP), monomethyl auristatin E (MMAE), 5-benzoyl valeric acid-AE ester (AEVB), tubulysins, disorazole, epothilones, SN-38, topotecan, rhizoxin, duocarmycin, actinomycin, ansamitocin-P3, duocarmycin, duocarmycin B2, maytansine, maytensinoids (DM1, DM2, DM3, DM4), calicheamicin, echinomycin, colchicine, estramustine, cemadotin, eleutherobin, 1-hydroxyauramycin A, aclacinomycin, abafilomycin C1, dinaktin, doxorubicin, morpholino-doxorubicin, cyanomorpholino-doxorubicin, dolastatin, dolestatin-10, combretastatin, leptomycin B, pluramycins, staurosporine, nogalamycin, rhodomycins, mithramycin, rabelomycin, rapamycin, alnumycin, chartreusin, geliomycin, gilvocarcin, piericidin, chlorambucil, cyclophosphamide, melphalan, cyclopropane, methotrexate, dichlorormethatrexate, methopterin, cytosine arabinoside, leurosine, leurosideine, mitomycin C, mitomycin A, carminomycin, aminopterin, tallysomycin, podophyllotoxin, camptothecin, cisplatin, carboplatin, metallopeptides containing platinum, copper, vanadium, iron, cobalt, gold, cadmium, gallium, iron zinc, and nickel and radionuclides.

18. The macromolecule according to claim 15, wherein said cytotoxic agent is selected from the group consisting of geldanamycin, auristatin, duocarmycin, maytansine, calicheamicin, doxorubicin, vinblastine, desacetyl vinblastine hydrazine, and daunorubicin.

19. The macromolecule according to claim 1, wherein at least one of said polymers (b) and/or (a) further comprises a detection marker.

20. The macromolecule according to claim 1 further comprising a targeting agent.

21. The macromolecule according to claim 20, wherein said targeting agent is an antibody, a vitamin, a hormone, a neurotransmitter, a protein, or a peptide.

22. The macromolecule according to claim 20, wherein said polymer (a) or (b) further comprises at least one detection marker.

23. The macromolecule according to claim 22, wherein said detection marker is selected from the group consisting of a fluorescein, a coumarin, a radionuclide, and a metal chelator carrying radionuclides.

24. A macromolecule biotin conjugate comprising
a. a macromolecule according to claim 1 and
b. at least one trifunctional cross-linking moiety bonded to said polymer (a), said trifunctional cross-linking moiety being coupled to a biotin.

25. The macromolecule biotin conjugate according to claim 24, wherein said trifunctional cross-linking moiety is selected from the group consisting of triaminobenzene, tricarboxybenzene, dicarboxyaniline and diaminobenzoic acid.

26-27. (canceled)

28. The macromolecule biotin conjugate according to claim 24, wherein linker I comprises a detection marker.

29-45. (canceled)
